(19) United States
(12) Patent Application Publication
(10) Pub. No.: US 2019/0345536 A1
(43) Pub. Date: Nov. 14, 2019

(54) DETECTION METHOD FOR CHROMOSOMAL ABNORMALITIES AND METHOD FOR EVALUATING INDUCIBILITY

(71) Applicants: Sumitomo Chemical Company, Limited, Tokyo (JP); Kyoto University, Kyoto-shi, Kyoto (JP)

(72) Inventors: Katsunori SASAKI, Chuo-ku, Tokyo (JP); Koichi SAITO, Osaka-shi, Osaka (JP); Shin KANEKO, Kyoto-shi, Kyoto (JP); Yohei KAWAI, Kyoto-shi, Kyoto (JP)

(21) Appl. No.: 16/474,170

(22) PCT Filed: Dec. 26, 2017
(86) PCT No.: PCT/JP2017/046756

§ 371 Date: Jun. 27, 2019

(30) Foreign Application Priority Data
Dec. 27, 2016 (JP) 2016-252673

(51) Int. Cl. C12Q 1/68; C12N 5/0783

(52) U.S. Cl. CPC C12Q 1/68; C12N 5/0636; C12N 2501/998; C12N 2506/02; C12N 2506/45

(57) ABSTRACT

A method for detecting chromosomal aberration including adding an anti-CD3 antibody to a human pluripotent stem cell-derived T lymphocyte and culturing the T lymphocyte, and detecting chromosomal aberration in the cultured T lymphocyte is described. Also described is a method for evaluating inducibility by a test substance of chromosomal aberration including adding an anti-CD3 antibody to a human pluripotent stem cell-derived T lymphocyte and culturing the T lymphocyte, adding a test substance to the cultured T lymphocyte, measuring frequency of chromosomal aberration in the T lymphocyte after addition of the test substance, and evaluating inducibility of chromosomal aberration by comparing the frequency of chromosomal aberration with the frequency standard value in the control group.

### TECHNICAL FIELD

The present invention relates to a method for detecting chromosomal aberration and a method for evaluating inducibility using human pluripotent stem cell-derived T lymphocytes.

### BACKGROUND ART

Chromosome aberration is one of the endpoints of genotoxicity, and one of the most important evaluation endpoints in predicting the effects by chemical substance and so on on the human health.

In genotoxicity test which is a safety test performed for evaluating genotoxicity, various tests relating to the parameters of, for example, DNA damage, gene mutation inducibility, chromosomal aberration inducibility and so on are utilized. The genotoxicity test includes an in vitro genotoxicity test performed in vitro using bacteria, cells, and so on, which is positioned as a screening test to easily and quickly grasp the genotoxicity of test substances or a test to understand the toxicity mode of action of the test substance, and an in vivo genotoxicity test using experimental animals, which is positioned as a conclusive test for final evaluation of genotoxicity. It is difficult to conduct in vivo genotoxicity tests on all the enormous number of chemical substances and so on both in terms of period and cost and also from the aspect of animal care. Thus, efficient use of in vitro genotoxicity tests has conventionally been demanded.

For evaluation of chromosomal aberration in vitro, an in vitro chromosomal aberration test, an in vitro micronucleus test, an in vitro comet assay for evaluation of damage to DNA constituting the chromosome, and so on are widely used.

While various cells can be utilized for evaluation of chromosomal aberration in vitro, mammalian-derived immortalized cells such as Chinese hamster lung-derived fibroblast cell lines CHL/IU and V79, Chinese hamster ovary-derived cell line CHO-K1, and so on are widely used because they are easy to handle and due to high detection sensitivity. In addition, it has been reported that human lymphoblastoid cell line TK6 which is a human immortalized cell and human lymphocyte (HuLy) derived from fresh blood which is a human biological sample are used (non-patent document 1).

### DOCUMENT LIST

### Non-Patent Documents

non-patent document 1: Fowler P., Smith K., Young J., Jeffrey L., Kirkland D., Pfuhler S., Carmichael P.: Reduction of misleading (“false”) positive results in mammalian cell genotoxicity assays. I. Choice of cell type. Mutation Research 742: p 11-25, 2012.
non-patent document 2: Kimura A., Miyata A., Honma M.: A combination of in vitro comet assay and micronucleus test using human lymphoblastoid TK6 cells. Mutagenesis 28(5): p 583-590, 2013.
non-patent document 3: Honma M., Hayashi M.: Comparison of In Vitro Micronucleus and Gene Mutation Assay Results for p53-Competent Versus p53-Deficient Human Lymphoblastoid Cells. Environmental and Molecular Mutagenesis 52: p 373-384, 2011.

### SUMMARY OF THE INVENTION

### Problems to be Solved by the Invention

However, it is known that false positive results occur when immortalized cells derived from mammal are used (non-patent document 1). As the cause thereof, the gene profile of cells which is different from that of the individual organism, as evidenced by dysfunction of cell cycle regulator p53 also known as a tumor-suppressor gene and so on, has been pointed out (non-patent document 2).

Furthermore, while human lymphoblastoid cell line TK6 is a human immortalized cell with the normal function of p53, it has chromosomal abnormalities such as trisomy of chromosome 13, translocation of chromosome 14 and chromosome 20, translocation of chromosome 3 and chromosome 21, and so on, and additionally shows genomic instability such as variation of chromosomal number due to passage culture for a long term. There is a research report suggesting that the function of p53 is not the only determinant of the occurrence of false positive in in vitro genotoxicity tests and the occurrence depends on the comprehensive profile of various genes and the animal species from which the cells are derived (non-patent document 3), and such abnormal cell TK6 cannot always show the result that accurately reflects the responsiveness in the human body.

When lymphocyte HuLy derived from human fresh blood is used, it is necessary to collect fresh human blood for every test since proliferation ability is limited, and therefore, it is difficult to ensure stable supply of cells by securing donors that meet various criteria such as age, medical history, smoking history, and so on. HuLy is known to show slightly different properties depending on the donor from which it is derived, that is, slightly different responsiveness and so on in the test. In addition, it is assumed that the cell profile varies depending on the donor's physiological condition at the time of collection even though the donor is the same. HuLy derived from fresh blood is limited in the number of cells that can be collected from the same donor. Therefore, it is difficult to carry out, in a standard manner, comparative experiments to examine difference in responsiveness among donors and homogeneous reproductive experiments using cells derived from the same donor.

Therefore, it is an object of the present invention to solve the above-mentioned problem associated with the conventional evaluation method of chromosomal aberration in vitro using immortalized cells derived from mammal, TK6, HuLy and so on, and provide a method for evaluating chromosomal aberration using a human normal cell type that can be supplied stably and homogeneously in a large amount.

### Means of Solving the Problems

To solve the aforementioned problems, the present inventors took note of pluripotent stem cells capable of proliferating almost unlimitedly and capable of differentiating into various cells, and obtained an idea of detecting and testing chromosomal abnormalities by using human pluripotent stem cell-derived T lymphocytes. However, when human iPS cell-derived T lymphocytes are used, stimulation of proliferation by PHA (phytohemagglutinin) used in the conventional methods by HuLy fails to cause sufficient division of the T lymphocytes. Thus, the detection and testing of chromosomal abnormalities cannot be performed. Then, the present inventors have conducted intensive studies and found that human iPS cell-derived T lymphocytes divide well when an anti-CD3 antibody is used as a proliferation stimulation factor, and the detection and testing of chromosomal abnormalities can be performed.

Accordingly, the present invention provides the following [1] to [14].

[1] A method for detecting chromosomal aberration comprising the following steps (1) and (2):
(1) a step of adding an anti-CD3 antibody to a human pluripotent stem cell-derived T lymphocyte and culturing the T lymphocyte,
(2) a step of detecting chromosomal aberration in the T lymphocyte cultured in step (1).
[2] The detection method of [1] wherein the human pluripotent stem cell is a human ES cell.
[3] The detection method of [1] wherein the human pluripotent stem cell is a human iPS cell.
[4] The detection method of any of [1] to [3] wherein step for detecting chromosomal aberration is a step for detecting chromosomal aberration by measuring frequency of micronucleus.
[5] The detection method of any of [1] to [3] wherein the step for detecting chromosomal aberration is a step for detecting chromosomal aberration by observation of chromosomal morphology.
[6] The detection method of any of [1] to [3] wherein the step for detecting chromosomal aberration is a step for detecting chromosomal aberration by evaluation of DNA damage.
[7] A detection kit for use in the detection method of any of [1] to [6], comprising an anti-CD3 antibody and a human pluripotent stem cell-derived T lymphocyte.
[8] A method for evaluating inducibility by a test substance of chromosomal aberration comprising the following steps (1)-(4):
(1) a step of adding an anti-CD3 antibody to a human pluripotent stem cell-derived T lymphocyte and culturing the T lymphocyte,
(2) a step of adding a test substance to the T lymphocyte cultured in step (1),
(3) a step of measuring frequency of chromosomal aberration in the T lymphocyte after addition of the test substance,
(4) a step of evaluating inducibility of chromosomal aberration by comparing the frequency of chromosomal aberration with the standard value.
[9] The method of [8] wherein the human pluripotent stem cell is a human ES cell.
[10] The method of [8] wherein the human pluripotent stem cell is a human iPS cell.
[11] The method of any of [8] to [10] wherein step (3) is a step of measuring frequency of chromosomal aberration in T lymphocyte after addition of a test substance by measuring frequency of micronucleus.
[12] The method of any of [8] to [10] wherein step (3) is a step of measuring frequency of chromosomal aberration in T lymphocyte after addition of a test substance by observation of chromosomal morphology.
[13] The method of any of [8] to [10] wherein step (3) is a step of measuring frequency of chromosomal aberration in T lymphocyte after addition of a test substance by evaluation of DNA damage.
[14] A test kit for use in the evaluation method of any of [8] to [13], comprising an anti-CD3 antibody and a human pluripotent stem cell-derived T lymphocyte.

### Effect of the Invention

According to the present invention, a human normal T lymphocyte capable of detecting and testing chromosomal aberration that can be supplied stably and homogeneously in a large amount can be provided. By detecting chromosomal aberration using the cell, the problems of conventional evaluation methods of chromosomal aberration in vitro using mammal-derived immortalized cell, TK6, HuLy and so on can be solved.

### BRIEF DESCRIPTION OF THE DRAWINGS

FIG. 1 shows a dose response relationship with regard to mitomycin C (MMC) obtained in an in vitro micronucleus test (Example 2) using human iPS cell-derived T lymphocytes. The horizontal axis shows the treatment dose of MMC, and the vertical axis shows, on the first axis for a bar graph, frequency of micronucleus (%) of each dose and, on the second axis for a line graph, RI (%) (Replication Index) as an index of the cytotoxicity of each dose. On the bar graph, ** is added to the dose that showed 1% significant increase in frequency of micronucleus by the Fisher's exact test.

FIG. 2 shows a dose response relationship with regard to cytosine arabinoside (AraC) obtained in an in vitro micronucleus test (Example 2) using human iPS cell-derived T lymphocytes. The horizontal axis shows the treatment dose of AraC, and the vertical axis shows, on the first axis for a bar graph, frequency of micronucleus (%) of each dose and, on the second axis for a line graph, RI (%) as an index of the cytotoxicity of each dose. On the bar graph, ** is added to the dose that showed 1% significant increase in frequency of micronucleus by the Fisher's exact test.

FIG. 3 shows a dose response relationship with regard to ethyl methanesulfonate (EMS) obtained in an in vitro micronucleus test (Example 2) using human iPS cell-derived T lymphocytes. The horizontal axis shows the treatment dose of EMS, and the vertical axis shows, on the first axis for a bar graph, frequency of micronucleus (%) of each dose and, on the second axis for a line graph, RI (%) as an index of the cytotoxicity of each dose. On the bar graph, ** is added to the dose that showed 1% significant increase in frequency of micronucleus by the Fisher's exact test.

FIG. 4 shows a dose response relationship with regard to bleomycin sulfate (BLM) obtained in an in vitro micronucleus test (Example 2) using human iPS cell-derived T lymphocytes. The horizontal axis shows the treatment dose of BLM, and the vertical axis shows, on the first axis for a bar graph, frequency of micronucleus (%) of each dose and, on the second axis for a line graph, RI (%) as an index of the cytotoxicity of each dose. On the bar graph, ** is added to the dose that showed 1% significant increase in frequency of micronucleus by the Fisher's exact test.

FIG. 5 shows a dose response relationship with regard to 1-methyl-3-nitro-1-nitrosoguanidine (MNNG) obtained in an in vitro micronucleus test (Example 2) using human iPS cell-derived T lymphocytes. The horizontal axis shows the treatment dose of MNNG, and the vertical axis shows, on the first axis for a bar graph, frequency of micronucleus (%) of each dose and, on the second axis for a line graph, RI (%) as an index of the cytotoxicity of each dose. On the bar graph, ** is added to the dose that showed 1% significant increase in frequency of micronucleus by the Fisher's exact test.

FIG. 6 shows a dose response relationship with regard to colchicine (COL) obtained in an in vitro micronucleus test (Example 2) using human iPS cell-derived T lymphocytes. The horizontal axis shows the treatment dose of COL, and the vertical axis shows, on the first axis for a bar graph, frequency of micronucleus (%) of each dose and, on the second axis for a line graph, RI (%) as an index of the cytotoxicity of each dose. On the bar graph, * is added to the dose that showed 5% significant increase and ** is added to the dose that showed 1% significant increase in frequency of micronucleus by the Fisher's exact test.

FIG. 7 shows a dose response relationship with regard to vinblastine (VBL) obtained in an in vitro micronucleus test (Example 2) using human iPS cell-derived T lymphocytes. The horizontal axis shows the treatment dose of VBL, and the vertical axis shows, on the first axis for a bar graph, frequency of micronucleus (%) of each dose and, on the second axis for a line graph, RI (%) as an index of the cytotoxicity of each dose. On the bar graph, ** is added to the dose that showed 1% significant increase in frequency of micronucleus by the Fisher's exact test.

FIG. 8 shows a dose response relationship with regard to cyclophosphamide monohydrate (CPA) obtained in an in vitro micronucleus test (Example 2) using human iPS cell-derived T lymphocytes. The horizontal axis shows the treatment dose of CPA, and the vertical axis shows, on the first axis for a bar graph, frequency of micronucleus (%) of each dose and, on the second axis for a line graph, RI (%) as an index of the cytotoxicity of each dose. As regards all doses, on the bar graph, ** is added to the dose that showed 5% significant increase in frequency of micronucleus by the Fisher's exact test.

FIG. 9 shows a dose response relationship with regard to D(−)-mannitol (MAN) obtained in an in vitro micronucleus test (Example 2) using human iPS cell-derived T lymphocytes. The horizontal axis shows the treatment dose of MAN, and the vertical axis shows, on the first axis for a bar graph, frequency of micronucleus (%) of each dose and, on the second axis for a line graph, RI (%) as an index of the cytotoxicity of each dose. As regards all doses, no statistically significant increase in frequency of micronucleus was observed.

FIG. 10 shows a dose response relationship with regard to mitomycin C (MMC) obtained in an in vitro chromosomal aberration test (Example 5) using human iPS cell-derived T lymphocytes. The horizontal axis shows the treatment dose of MMC, and the vertical axis shows, on the first axis for a bar graph, frequency of chromosomal aberration (%) of each dose and, on the second axis for a line graph, RICC (%) (Relative increase in cell count) as an index of the cytotoxicity of each dose. On the bar graph, moreover, ** is added to the dose that showed 1% significant increase in frequency of chromosomal aberration by the Fisher's exact test.

FIG. 11 shows a dose response relationship with regard to ethyl methanesulfonate (EMS) obtained in an in vitro chromosomal aberration test (Example 5) using human iPS cell-derived T lymphocytes. The horizontal axis shows the treatment dose of EMS, and the vertical axis shows, on the first axis for a bar graph, frequency of chromosomal aberration (%) of each dose and, on the second axis for a line graph, RICC (%) as an index of the cytotoxicity of each dose. On the bar graph, moreover, ** is added to the dose that showed 1% significant increase in frequency of chromosomal aberration by the Fisher's exact test.

FIG. 12 shows a dose response relationship with regard to D(−)-mannitol (MAN) obtained in an in vitro chromosomal aberration test (Example 5) using human iPS cell-derived T lymphocytes. The horizontal axis shows the treatment dose of MAN, and the vertical axis shows, on the first axis for a bar graph, frequency of chromosomal aberration (%) of each dose and, on the second axis for a line graph, RICC (%) as an index of the cytotoxicity of each dose. As regards all doses, no statistically significant increase in frequency of chromosomal aberration was observed.

FIG. 13 shows a dose response relationship with regard to ethyl methanesulfonate (EMS) obtained in an in vitro comet assay (Example 6) using human iPS cell-derived T lymphocytes. The horizontal axis shows the treatment dose of EMS, and the vertical axis shows, on the first axis for a bar graph, mean value of the median of the % tail DNA at each dose in triplicate and, on the second axis for a line graph, RCC (%) (Relative cell count) as an index of the cytotoxicity of each dose. On the bar graph, ** is added to the dose that showed 1% significant increase in % tail DNA by the Dunnett's test.

FIG. 14 shows a dose response relationship with regard to D(−)-mannitol (MAN) obtained in an in vitro comet assay (Example 6) using human iPS cell-derived T lymphocytes. The horizontal axis shows the treatment dose of MAN, and the vertical axis shows, on the first axis for a bar graph, mean value of the median of the % tail DNA at each dose in triplicate and, on the second axis for a line graph, RCC (%) as an index of the cytotoxicity of each dose. As regards all doses, no statistically significant increase in % tail DNA was observed.

### DESCRIPTION OF EMBODIMENTS

The description of embodiments for carrying out the present invention is explained in detail below.

### Explanation of Common Terms

In the present specification, the “genotoxicity” means the potential that causes irreversible changes in the genetic information of an organism, such as the number and structure of chromosomes or the DNA constituting the chromosome, namely, mutation.

In the present specification, the “false positive” means a positive reaction with poor biological significance, which is observed only in in vitro tests even though chromosomal abnormalities are not induced in vivo. When a positive reaction is found in an in vitro test, it is essential to perform an in vivo test to confirm the presence or absence of inducibility of chromosomal aberration in vivo. Frequent occurrence of false positive results increases the number of unnecessary in vivo tests. That is, from various aspects of evaluation period, cost, animal care and so on, it is required to decrease false positive results in in vitro tests.

In one embodiment, the present invention provides a method for detecting chromosomal aberration including

(1) a step of adding an anti-CD3 antibody to a human pluripotent stem cell-derived T lymphocyte and culturing the T lymphocyte, and
(2) a step of detecting chromosomal aberration in the T lymphocyte cultured in step (1) (hereinafter sometimes to be abbreviated as “the detection method of the present invention”).

In the present invention, the “stem cell” means a cell having an ability to divide and make the same cell as the original cell, that is, self-renewal ability and an ability to differentiate into a different type of cell, and capable of proliferating continuously.

The “pluripotent stem cell” in the present invention means a stem cell that permits cultivation in vitro, and having the potential for differentiating into tissues derived from the three germ layers (ectoderm, mesoderm and endoderm), namely, pluripotency. The “pluripotent stem cell” can be established from a fertilized egg, a cloned embryo, a germ stem cell, or a stem cell in tissue. More specific examples of the “pluripotent stem cell” in the present invention include embryonic stem cells (ES cells), induced pluripotent stem cells (iPS cells) induced from somatic cells, embryonic carcinoma cells (EC cells), and embryonic germ cells (EG cells) and so on, and preferred is ES cell or iPS cell.

In the present invention, the “ES cell” is a stem cell having a self-renewal ability and pluripotency and means a pluripotent stem cell derived from an early embryo. ES cell was established for the first time in 1981 and has also been applied to generate knockout mouse from 1989. In 1998, human ES cell was established and has also been utilized to regenerative medicine.

In the present invention, the “iPS cell” is a pluripotent stem cell induced from a somatic cell and means a cell artificially imparted with pluripotency similar to that of embryonic stem cell by reprogramming the somatic cell. Specifically, an induced pluripotent stem cell with pluripotency established by reprogramming differentiated cells such as fibroblast and so on by expression of genes such as Oct3/4, Sox2, Klf4, Myc and so on can be mentioned. In 2006, Yamanaka et al. established an induced pluripotent stem cell from mouse fibroblasts (Cell, 2006, 126(4), p 663-676). In 2007, they established an induced pluripotent stem cell having pluripotency similar to that of embryonic stem cells from human fibroblasts (Cell, 2007, 131(5), p 861-872; Science, 2007, 318(5858), p 1917-1920; Nat Biotechnol., 2008, 26(1), p 101-106). The iPS cell to be used in the present invention may be produced from somatic cell by a method known per se, or may be an already established and stocked iPS cell. The somatic cell from which the iPS cell to be used in the present invention derives is not limited, and from the aspect of induction efficiency into T lymphocytes, preferred is T lymphocyte. Human T lymphocyte can be isolated from a human tissue by a known method. Examples of the human tissue include peripheral blood, lymph node, bone marrow, thymus, spleen, cord blood, lesion tissue, and the human tissue is not particularly limited as long as it is a tissue containing the aforementioned T lymphocyte.

In the present invention, the “T lymphocyte” means one type of lymphocyte, also called T cell, found in lymphatic organs or peripheral blood, etc., and is characterized by differentiation and maturation mainly in the thymus and expressing T cell receptors (TCRs). T lymphocytes are further classified into multiple cell types based on their functions and known main cell types include helper T cell, killer T cell, natural killer T (NKT) cell and so on. T lymphocytes are frequently used for various experiments and detection systems for examining responses in human cells, including genotoxicity tests since they are human normal cells that can be collected from human peripheral blood, and can be easily proliferated by various growth factors. Examples of the human T lymphocyte usable in the present invention include helper T cell which is CD4 positive cell, killer T cell which is CD8 positive cell, naive T cell (CD45RA⁺CD62L⁺ cell), central memory T cell (CD45RA⁻CD62L⁺ cell), effector memory T cell (CD45RA⁻CD62L⁺ cell) and so on, and cells in the process of differentiation into these cells are also included. Preferred T lymphocyte is CD8SP (Single Positive) killer T cell.

In the present invention, the “human pluripotent stem cell-derived T lymphocyte” means T lymphocyte produced by inducing differentiation of human pluripotent stem cell. As a method for inducing differentiation of human pluripotent stem cell into T lymphocyte, it can be appropriately selected from various known differentiation induction methods and used. Examples of the known differentiation induction method include a method for inducing T lymphocyte from human ES cell described in “Timmermans, F. et al., J. Immunol., 2009, 182, 68879-6888”. In addition, for example, human iPS cell established from human peripheral blood T lymphocyte can be induced to differentiate into T lymphocyte by the method described in “Nishimura, T. et al. Cell Stem Cell, 2013, 12, 114-126”. When pluripotent stem cell-derived T lymphocyte is used for the evaluation method of the present invention, T lymphocyte may be produced from pluripotent stem cell and immediately used for the evaluation, or T lymphocyte produced in advance and stocked by cryopreservation and so on (hereinafter sometimes to be abbreviated as “stock cell”) may be used. It is preferable that the quality of the stock cell has been evaluated in advance by expression of cell specific marker, evaluation of the frequency of chromosomal aberration and proliferation ability or others. From the aspects of convenience and reproducibility, it is preferable to use stock cells that have been evaluated for quality.

Chromosomal aberration of the cell is fixed on cell division, and therefore, the pluripotent stem cell-derived T lymphocyte used for the evaluation method of the present invention is required to have good proliferation ability. The proliferation ability of the cell can be calculated, for example, from CBPI (Cytokinesis-block proliferation index) shown by the following formula which is an index showing the degree of cell division in the treatment period with CytoB, in which a larger numerical value indicates more active division of the cells. CBPI is 1 when all the observation target cells did not divide, and 2 when all the observation target cells underwent one division process. CBPI is specified in the test guideline (OECD guidelines for the testing of chemicals 487: In vitro mammalian cell micronucleus test, adopted 26 Sep. 2014) as an index to evaluate cytostasis by a test substance when performing an in vitro micronucleus test by adding CytoB. For the detection of micronucleus or chromosomal aberration, division of the observation target cell is essential, and when performing an in vitro micronucleus test by adding CytoB, binucleated cell is the observation target. Therefore, detection and testing of chromosomal aberration require the cells to be in good division state and CBPI to show high values. CBPI depends on the treatment time with CytoB, and thus there is no clear consensus on the standard value to secure a good division state. Generally, when it is not less than 1.5, the cells are sufficiently practical as a detection and test system.

$$CBPI = \frac{( \begin{matrix}mononucleated cell number + 2 \times binucleated cell number + \\ 3 \times multinucleated cell number\end{matrix} )}{total observed cell number}$$

The pluripotent stem cell-derived T lymphocyte used for the evaluation method of the present invention is desirably evaluated in advance that it shows an appropriate response to a known compound that induces chromosomal aberration. Examples of the aforementioned known compound include, but are not limited to, mitomycin C (MMC) (e.g., CAS No. 50-07-7, Kyowa Hakko Kirin Co., Ltd.), cytosine arabinoside (AraC) (e.g., CAS No. 147-94-4, nacalai tesque), ethyl methanesulfonate (EMS) (e.g., CAS No. 62-50-0, nacalai tesque), bleomycin sulfate (BLM) (e.g., CAS No. 9041-93-4, LKT laboratories) and 1-methyl-3-nitro-1-nitrosoguanidine (MNNG) (e.g., CAS No. 70-25-7, nacalai tesque) and so on, which are micronucleus inducible compounds due to chromosomal structural aberration, colchicine (COL) (e.g., CAS No. 64-86-8, Wako) and vinblastine sulfate (VBL) (e.g., CAS No. 143-67-9, Wako) and so on, which are micronucleus inducible compounds due to numerical chromosomal aberration, cyclophosphamide monohydrate (CPA) (e.g., CAS No. 6055-19-2, nacalai tesque) and so on, which is a compound that induces micronucleus after metabolic activation by metabolic enzymes in vivo, D(−)-mannitol (MAN) (e.g., CAS No. 69-65-8, Wako), Triton X-100 and so on which are compounds that do not induce micronucleus. All of the aforementioned compounds are compounds whose reactivity (chromosomal aberration inducibility) has been sufficiently evaluated by a large number of known documents and so on (e.g., Mutat Res, 2006, 607, p 37-60, Mutagenesis, 2011, 26(6), p 763-770 and so on).

In the present invention, as the “step of adding an anti-CD3 antibody to a human pluripotent stem cell-derived T lymphocyte and culturing the T lymphocyte” (“step of applying proliferation stimulation to T lymphocyte”), a step of adding an anti-CD3 antibody to a medium and culturing the T lymphocyte can be mentioned. By applying proliferation stimulation, a cell aggregate can be formed. By observing that a plurality of cell aggregates are formed, it can be confirmed that a proliferation stimulation has been applied. To evaluate the cell cycle dependency of the action of a test substance, the T lymphocyte in the quiescent phase without the proliferation stimulation may be treated with a test substance and then subjected to the proliferation stimulation. Generally, various factors such as various lectin molecules including PHA are known as proliferation stimulation factors for T lymphocytes. However, it is essential to use an anti-CD3 antibody as a proliferation stimulation factor of human pluripotent stem cell-derived T lymphocyte in the present invention, as shown in the below-mentioned Examples. That is, the proliferation stimulation using PHA used in the conventional method by HuLy cannot afford observation of human iPS cell-derived T lymphocytes which have divided with sufficient frequency, irrespective of the presence or absence of coculture with feeder cells, and the cells are not practical as detection or testing. On the other hand, when an anti-CD3 antibody is used as a proliferation stimulation factor, the detection and testing become possible.

While many clones exist for the anti-CD3 antibody to be used for proliferation stimulation, for example, “OKT3” clone can be used. When proliferation stimulation is applied to the T lymphocyte by using an anti-CD3 antibody, antibodies such as anti-CD28 antibody, anti-CD2 antibody and so on, and cytokines such as IL-2, IL-7, IL-15 and so on may be further added.

Various known media for cell culture can be appropriately selected and used as the medium to which the proliferation stimulation factor is added. Specific examples of the medium suitable for culturing T lymphocytes include RPMI1640 medium. Besides the proliferation stimulation factor, various sera (e.g., human serum, fetal bovine serum), amino acids (e.g., L-glutamine), antibiotics (e.g., penicillin, streptomycin), other cytokine and so on may be added to the medium before use.

The anti-CD3 antibody to be used for proliferation stimulation may be bonded to magnetic bead and so on, or instead of adding the aforementioned anti-CD3 antibody into the medium, stimulation may be given by culturing the T lymphocytes for a given period on a culture dish on which the anti-CD3 antibody is bound to the surface.

While the concentration of the anti-CD3 antibody to be added to the medium is not particularly limited, it is preferably 1 to 10 times the cell density of the T lymphocytes described later. While the concentration of the anti-CD3 antibody bound on a culture dish to stimulate proliferation of the T lymphocytes is not particularly limited, the concentration during coating is preferably 0.1-100 μg/mL, more preferably 0.5-50 μg/mL. In one embodiment of the present invention, the concentration of anti-CD3 antibody during coating is 0.5-1.0 μg/mL.

The seeding density of the T lymphocytes when applying proliferation stimulation with anti-CD3 antibody is not particularly limited. To increase contact opportunities for T lymphocytes to promote clustering and provide efficient proliferation stimulation, it is preferable to perform high density culture. Specifically, not less than 1×10⁵ cells/mL is preferable, not less than 5×10⁵ cells/mL is more preferable, and not less than 1×10⁶ cells/mL is particularly preferable.

The “seeding density of the T lymphocytes when applying proliferation stimulation with anti-CD3 antibody” here means the final cell density of T lymphocytes after seeding T lymphocytes (hereinafter sometimes to be referred to as “final cell density”).

The culture period after addition of the anti-CD3 antibody is not particularly limited as long as it is sufficient for applying proliferation stimulation with anti-CD3 antibody. It is, for example, not less than 6 hr, preferably not less than 12 hr.

Long-term sustained proliferation stimulation with an anti-CD3 antibody may induce activation induced cell death. To avoid this, the anti-CD3 antibody may be removed from the culture system after a certain period of time after the start of the proliferation stimulation. While the period from the start of proliferation stimulation to removal is not particularly limited, it is preferably within 72 hr, more preferably within 24 hr.

After culturing for a sufficient period of time to promote T lymphocyte proliferation, the cell density can also be reduced along with the removal of anti-CD3 antibody to prevent the continuation of proliferation stimulation due to the interaction between cells. The cell density at this time is not particularly limited as long as cell proliferation is possible to such an extent that detection and testing of chromosomal aberration is possible. It is preferably 1×10⁵-1×10⁶ cells/mL, more preferably 1×10⁵-6×10⁵ cells/mL. In one embodiment of the present invention, the cells are cultured at a density of 4×10⁵ cells/mL.

While the culture period from the removal of the anti-CD3 antibody to the addition of the test substance is also not particularly limited as long as the above-mentioned object can be achieved, it is preferably within 48 hr, more preferably 3-24 hr.

The pH of the medium is preferably about 6-about 8, and the CO₂ concentration is preferably about 2-5%. The culture temperature is generally about 30-40° C., preferably about 37° C. Where necessary, aeration and stirring may also be performed.

In the present invention, examples of the method for “detecting chromosomal aberration” include, but are not limited to, a method for detecting chromosomal aberration by measuring frequency of micronucleus, a method for detecting chromosomal aberration by observation of chromosomal morphology, a method for detecting chromosomal aberration by evaluation of DNA damage and so on.

In the present invention, the “micronucleus” means an abnormal nucleic acid structure that is smaller than the main nucleus, which is found in the cytoplasm of a cell in mitotic anaphase to interphase or quiescent phase. The micronucleus is known to be caused by an acentric chromosomal fragment originated from structural aberration of chromosome or whole chromosome that could not move to the pole due to abnormality of chromosomal segregation in mitotic phase, and becomes an index of chromosomal aberration developed during mitosis. The micronuclear frequency, namely, the proportion of cells with micronucleus in all cells to be the observation target, shows a frequency of occurrence of structural or numerical aberration in chromosome due to cell division during a certain culture period. That is, chromosomal aberration that has occurred during cell division can be detected by measuring the frequency of micronucleus using the procedure of the below-mentioned in vitro micronucleus test.

In the present invention, the procedure of an in vitro micronucleus test can be utilized for the detection of chromosomal aberration by “frequency of micronucleus”. The in vitro micronucleus test is a genotoxicity test to measure the frequency of nucleus-like structures smaller than the main nucleus, namely, micronucleus, in the cytoplasm of a cell in mitotic anaphase to interphase. The micronucleus is known to be caused by an acentric chromosome fragment originated from structural aberration of chromosome or whole chromosome that could not move to the pole due to abnormality of chromosomal segregation in mitotic phase, and becomes an index of chromosomal aberration developed during mitosis. The in vitro micronucleus test is being widely used for registration applications and regulation purposes as a test that can evaluate the ability of a test substance to induce chromosomal aberration with the same sensitivity as in in vitro chromosomal aberration tests. The standard test method is specified in the test guideline of OECD (OECD guidelines for the testing of chemicals 487: In vitro mammalian cell micronucleus test, adopted 26 Sep. 2014). The method is not limited to this test method and various known methods can be utilized.

In the present invention, “observation of chromosomal morphology” is an analysis method for evaluating whether there are structural or numerical aberrations in the chromosomes of cells of interest, that is, chromosomal abnormalities, the method including culturing the cells of interest for a given period, preparing a chromosome sample from cells in mitotic metaphase enriched by adding a mitosis inhibitor such as colcemid, applying a staining to chromosomes in one cell, and observing each chromosome under a microscope. The frequency of chromosomal aberration, namely, the proportion of cells having chromosomal aberration in all cells in mitotic metaphase to be the observation target shows a frequency of occurrence of structural or numerical aberration in chromosome due to cell division during a certain culture period. That is, chromosomal aberration that has occurred during cell division can be detected by measuring the frequency of chromosomal aberration by utilizing the below-mentioned procedure of an in vitro chromosomal aberration test.

In the present invention, the procedure of an in vitro chromosomal aberration test can be utilized for the detection of chromosomal aberration by “observation of chromosomal morphology”. The in vitro chromosomal aberration test is a genotoxicity test to measure the frequency of chromosomal aberration such as cleavage of chromosome and so on by observing the chromosomal morphology of cells in mitotic metaphase under a microscope. The standard test method is specified in the test guideline of OECD (OECD guidelines for the testing of chemicals 473: In vitro mammalian chromosomal aberration test, adopted 26 Sep. 2014). The chromosomal aberration inducibility of a test substance can be evaluated by comparing the frequency of chromosomal aberration observed in the cells treated with the test substance with that of the negative control group, i.e., untreated group. The method is not limited to this test method and various known methods can be utilized.

In the present invention, the “evaluation of DNA damage” means an analysis method including culturing the cells of interest for a given period and evaluating whether DNA of the cells of interest is damaged. For example, a specimen is prepared by embedding cells of interest cultured for a given period in agarose gel etc. and subjecting them to electrophoresis under alkaline conditions, the nucleic acid is fluorescently stained, each nucleus is observed under a microscope, and the proportion of the fluorescence intensity of the tail to the fluorescence intensity of the whole cell nucleus (% tail DNA), and so on can be used as an index value of the DNA damage.

The index value such as % tail DNA and so on in the nuclei to be the observation target becomes a quantitative index of damage such as cleavage and so on that occurs in the DNA constituting the chromosomes of cells in a given culture period. That is, DNA damage can be detected as chromosomal aberration by measuring the % tail DNA by utilizing the procedure of the below-mentioned in vitro comet assay. Alternatively, DNA damage may be evaluated as chromosomal aberration by detecting and analyzing expression of a DNA double strand cleavage marker (e.g., phosphorylated histone H2AX (γH2AX)) by immunostaining, Western blotting and so on using the marker as an index of genotoxicity. In addition, DNA damage may be evaluated as chromosomal aberration by directly detecting and analyzing a DNA adduct by utilizing instrument analysis by a mass analyzer and so on or labeling with radioisotope and using production of the DNA adduct as an index of genotoxicity.

Alternatively, DNA damage may be evaluated as chromosomal aberration by quantifying the degree of a DNA damage response (e.g., unscheduled DNA synthesis (UDS) response) as an index of genotoxicity (e.g., quantification of UDS response by determining the uptake of tritium-labeled thymidine by autoradiography).

In the present invention, the procedure of an in vitro comet assay can be utilized for detection of chromosomal aberration by “evaluation of DNA damage”. The in vitro comet assay is also called single cell gel electrophoresis (SCGE) and is a genotoxicity test that detects a DNA fragment resulting from DNA damage by electrophoresing the cell nucleus under alkaline conditions and observing the shape of cell nucleus under nucleic acid staining. DNA inherently has a negative charge and has the property of moving to the anode side in the presence of an electric field. Undamaged DNA hardly moves even under electrophoresis since it has a higher-order structure, and the nucleus maintains a circular shape. On the other hand, a DNA fragment resulting from DNA damage does not have a higher-order structure and moves to the anode side in the presence of an electric field. As a result, the nucleus with DNA damage takes a comet-like structure (comet) with a tail according to the degree of the damage. That is, DNA damaging activity of a test substance can be estimated by electrophoresing the nucleus after a treatment with the test substance, and measuring the index value such as the proportion of the fluorescence brightness of the tail to the fluorescence brightness of the whole cell nucleus (% tail DNA), and so on under fluorescent staining of the nucleic acid. In vitro comet assay does not have an internationally-standardized protocol like the OECD test guideline. However, it can be performed according to the method described in various known documents (e.g., Kimura A., Miyata A., Honma M.: A combination of in vitro comet assay and micronucleus test using human lymphoblastoid TK6 cells. Mutagenesis 28(5): p 583-590, 2013, and so on). In the present invention, the procedure of the in vitro comet assay is not particularly limited. It can be performed according to various known methods (e.g., Mutagenesis, 2013, 28(5): p 583-590 and so on).

More specific embodiments of the method for detecting chromosomal aberration by measuring frequency of micronucleus, the method for detecting chromosomal aberration by observation of chromosomal morphology, the method for detecting chromosomal aberration by evaluation of DNA damage are explained in more detail in the following “The evaluation method of the present invention”.

In another embodiment, the present invention provides a method for evaluating chromosomal aberration inducibility by a test substance including the following steps (1)-(4):

(1) a step of adding an anti-CD3 antibody to a human pluripotent stem cell-derived T lymphocyte and culturing the T lymphocyte,
(2) a step of adding a test substance to the T lymphocyte cultured in step (1),
(3) a step of measuring frequency of chromosomal aberration in the T lymphocyte after addition of the test substance,
(4) a step of evaluating inducibility of chromosomal aberration by comparing the frequency of chromosomal aberration with the standard value
(hereinafter sometimes to be abbreviated as “the evaluation method of the present invention”).

Step (1) can be performed similarly to the above-mentioned “detection method of the present invention”.

Steps (2), (3) and (4) can be performed according to the various “methods for detecting chromosomal aberration” exemplified in the above-mentioned “detection method of the present invention”. Various methods are specifically described below.

### (A)<Detection of Chromosomal Aberration by Measuring Frequency of Micronucleus>

In the present invention, the procedure of an in vitro micronucleus test can be utilized for the detection of chromosomal aberration by “frequency of micronucleus”. For example, a method for detecting chromosomal aberration including

(A1) a step of applying proliferation stimulation to human pluripotent stem cell-derived T lymphocyte by using the aforementioned anti-CD3 antibody,
(A2) a step of adding a test substance or a vehicle to be used as a negative control to the T lymphocyte applied with the proliferation stimulation to perform a test substance treatment,
(A3) a step of recovering the cells after the test substance treatment, substituting the medium with a cell fixation solution to fix the cells, preparing the cell suspension after fixation to a cell density at which the suspension becomes slightly cloudy, adding dropwise the suspension on a slide glass and air drying same to prepare a specimen of the micronucleus, and
(A4) a step of staining the specimen of the micronucleus, observing the cells under a microscope provided with an optical system compatible with the staining method to measure frequency of micronucleus, and comparing the frequency of micronucleus of the test substance treatment group with that of the negative control group, i.e., a vehicle treatment group (untreated group) to evaluate micronucleus inducibility of the test substance, namely, chromosomal aberration inducibility, can be mentioned.

Step (A1) can be performed similarly to the above-mentioned “detection method of the present invention”.

### (A2) Step of Performing Test Substance Treatment

The treatment period of the test substance is not particularly limited, and also depends on the cell cycle of the T lymphocyte. It is typically 3 to 6 hr as a short time treatment and 20 to 72 hr as a long time treatment. Under the short time treatment conditions, generally, after the treatment is completed, the test substance treatment solution is replaced with a medium and the culture is continued until preparation of the specimen. A metabolic activation factor may be added at the time of test substance treatment to detect a test substance that induces chromosomal aberration or micronucleus through metabolic activation by metabolic enzymes. Examples of the widely-used metabolic activation factor include, but are not limited to, S9mix obtained by adding coenzyme etc. to S9 which is derived from a rat liver homogenate. The concentration of the added S9mix is not particularly limited, but is preferably used at a concentration that does not show significant toxicity to or micronucleus induction in the cells. The concentration is preferably 1-10% (v/v).

For the purpose of obtaining the information relating to cell division kinetics, and an index suggesting abnormality in the chromosome other than micronucleus such as NPB, NBUD and so on, an inhibitor of cytokinesis may be added before recovery of the cells. The inhibitor of cytokinesis is used at a concentration and a treatment time that does not cause significant toxicity or micronucleus inducibility of the cell. As the inhibitor of cytokinesis, 3-6 μg/mL of cytochalasin B (CytoB) dilution solution is often used, but the inhibitor is not limited thereto. The treatment time with CytoB is not particularly limited and depends on the division kinetics of T lymphocyte. It is, for example, 18-48 hr.

### (A3) Step of Preparing Micronucleus Specimen

The composition of the cell fixation solution used for preparation of a specimen of the micronucleus is not particularly limited, and ethanol, methanol, a mixture of ethanol and acetic acid, a mixture of methanol and acetic acid, a dilution solution of paraformaldehyde and so on can be mentioned.

Prior to fixation, a hypotonic treatment may be performed to favorably maintain the cytoplasm and facilitate observation. The composition of the hypotonic solution is not particularly limited and, for example, 75 mM KCl solution and so on can be mentioned.

### (A4) Measurement of Micronucleus Abnormality Frequency

The method for staining a specimen of the micronucleus is not particularly limited and, for example, double staining method of nucleus and cytoplasm with acridine orange and Giemsa staining method, nuclear staining method utilizing DAPI (2-(4-amidinophenyl)-1H-indole-6-carboxamidine), Hoechst 33342, Hoechst 33258, PI (Propidium Iodide), ethidium bromide, SYBR (registered trade mark) Gold, SYBR (registered trade mark) Green or other nucleic acid staining reagents, and so on can be mentioned.

For the purpose of evaluating the micronucleus induction mechanism and so on, kinetochore may be visualized by staining with a fluorescent in situ hybridization (FISH) method using a centromeric probe or an immunostaining method using an anti-kinetochore antibody.

While the number of cells to be observation object is not particularly limited, not less than 500 cells per one condition are preferable and not less than 1000 cells per one condition are more preferable, to ensure statistical accuracy. The observation step of the stained specimen can also be automated by using an image analyzer such as imaging cytometer and so on.

In addition, to facilitate testing of many samples, it is also possible to fix the cells directly on a culture vessel such as a 96-well plate without collecting the cells and prepare a specimen of micronucleus on the surface of the culture vessel. Using the aforementioned image analyzer, high-throughput automatic analysis can be performed. As an application example of the image analyzer, “Mutat Res 2013, 751(1), p 1-11” and so on have been reported but it is not limited thereto.

Alternatively, it is also possible to appropriately stain the recovered cell suspension and automatically analyze a large number of cells by using a cell analyzer in a flow path system such as a flow cytometer or a laser scanning cytometer. As a utilization example of the flow cytometer, “Mutat Res 2010, 703(2), p 191-199” and so on have been reported but it is not limited thereto.

When determining micronucleus inducibility by a test substance, various statistical tests can be performed as to the frequency of micronucleus between the test substance group and the negative control group. It can also be determined by the comparison of the range of the past achievements of frequency of micronucleus in the negative control group (historical control data) with the frequency of micronucleus in the test substance group. While the statistical test method to be used is not particularly limited, pair comparison by Fisher's exact test and a test of increase tendency by Cochran-Armitage tendency test are frequently used.

The frequency of micronucleus can be calculated by, for example, dividing the number of binucleated cells with micronucleus by the total number of binucleated cells. When the frequency of micronucleus is compared, for example, the presence or absence of a significant increase with significance probability (two-sided, 5% or 1%) may be tested by the Fisher's exact test between the frequency of micronucleus in a test substance treatment group and the frequency of micronucleus in the corresponding negative control group.

As an index of the cytostasis by a test substance, namely, cytotoxicity, for example, CBPI is calculated in the same manner as described above and RI (Replication index) can be calculated from the following formula:

$$R I ( \% ) = \frac{( CBPI in test substance \text{-} treated group - 1 )}{( CBPI in negative control group - 1 )} \times 100$$

When chromosomal aberration inducibility is evaluated using RI, for example, for respective test substances, the case when a 5% significant increase is not found in frequency of micronucleus at all treatment doses of RI of not less than 40% can be judged as negative, and the case when a 5% significant increase is found in frequency of micronucleus at a treatment dose of RI of not less than 40% can be judged as positive. In this case, a treatment dose of RI of less than 40% may be excluded from the determination of the chromosomal aberration inducibility because an increase in the frequency of micronucleus with low biological significance is likely to be found due to cytotoxicity. This method is an example of an evaluation method of chromosomal aberration inducibility, and the method is not limited thereto.

### (B)<Method for Detecting Chromosomal Aberration by Observation of Chromosomal Morphology>

In the present invention, the procedure of an in vitro chromosomal aberration test can be utilized for the detection of chromosomal aberration by “observation of chromosomal morphology”. For example, a method including

(B1) a step of applying proliferation stimulation to human pluripotent stem cell-derived T lymphocyte by using the aforementioned anti-CD3 antibody,
(B2) a step of adding a test substance or a vehicle to be used as a negative control to the T lymphocyte applied with the proliferation stimulation to perform a treatment with a test substance, adding a mitosis inhibitor before completion of the treatment and culturing for a given period to enrich the cells in mitotic metaphase,
(B3) a step of recovering the cells after the treatment, substituting the medium with a cell fixation solution to fix the cells, preparing the cell suspension after fixation to a cell density at which the suspension becomes slightly cloudy, adding dropwise the suspension on a slide glass and air drying same to prepare a chromosome specimen, and
(B4) a step of staining the specimen of the chromosomes, observing the cells under a microscope provided with an optical system compatible with the staining method to measure frequency of chromosomal aberration, and comparing the frequency of chromosomal aberration of a test substance treatment group with that of the negative control group, i.e., a vehicle treatment group (untreated group) to evaluate chromosomal aberration inducibility of a test substance can be mentioned.

Step (B1) can be performed similarly to the above-mentioned “detection method of the present invention”.

### (B2) Step of Performing Test Substance Treatment

The treatment period of the test substance is not particularly limited, and also depends on the cell cycle of the T lymphocyte. It is typically 3 to 6 hr as a short time treatment and 20 to 72 hr as a long time treatment. Under the short time treatment conditions, generally, after the treatment is completed, the test substance treatment solution is replaced with a medium and the culture is continued until preparation of the specimen. A metabolic activation factor may be added at the time of test substance treatment to detect a test substance that induces chromosomal aberration or micronucleus through metabolic activation by metabolic enzymes. Examples of the widely-used metabolic activation factor include, but are not limited to, S9mix obtained by adding coenzyme etc. to S9 which is derived from a rat liver homogenate. The addition concentration of the S9mix is not particularly limited, but is preferably 1-10% (v/v). As a mitosis inhibitor that enriches a cell in mitotic metaphase, 0.10 μg/mL colcemid dilution solution is often used, but it is not limited thereto. The treatment time with a mitosis inhibitor depends on the cell cycle of T lymphocyte and is not particularly limited. It is typically 1-2 hr.

### (B3) Step of Preparing Chromosome Specimen

As the cell fixation solution used for preparation of a chromosome specimen, a mixture of methanol:acetic acid=3:1(v/v) is frequently used; however, the solution is not limited thereto. Specific examples include ethanol, methanol, a mixture of ethanol and acetic acid, a mixture of methanol and acetic acid, a dilution solution of paraformaldehyde and so on. Prior to fixation, a hypotonic treatment may be performed to cause swelling of the cell to facilitate observation. The composition of the hypotonic solution is not particularly limited and, for example, 75 mM KCl solution and so on can be mentioned.

### (B4) Step of Measuring Frequency of Chromosomal Aberration

While the Giemsa staining method is often used as a method for staining a chromosome specimen, the method is not limited thereto and various nucleic acid staining reagents can be used. For the purpose of detecting chromosomal aberration in more detail, G band method or Q band method, the FISH method using a chromosome-specific DNA probe and so on may be used to perform staining capable of distinguishing individual chromosomes.

While the number of cells in mitotic metaphase to be the observation object is not particularly limited, not less than 100 cells in mitotic metaphase per 1 condition are generally observed to ensure statistical accuracy.

A part of the observation step of the stained specimen can also be semi-automated using an image analyzer. When determining chromosomal aberration inducibility by a test substance, various statistical tests can be performed as to the frequency of chromosomal aberration between the test substance group and the negative control group. It can also be determined by the comparison of the range of the past achievements of frequency of chromosomal aberration in the negative control group (historical control data) with the frequency of chromosomal aberration in the test substance group. While the statistical test method to be used is not particularly limited, pair comparison by Fisher's exact test and a test of increase tendency by Cochran-Armitage tendency test are frequently used.

The frequency of chromosomal aberration can be calculated by, for example, dividing the number of cells in mitotic metaphase with some chromosomal aberration by the total number of observed cells in mitotic metaphase. For example, the presence or absence of a significant increase with significance probability (two-sided, 5% or 1%) may be tested by the Fisher's exact test between the frequency of chromosomal aberration in a test substance treatment group and the frequency of chromosomal aberration in the corresponding negative control group.

As an index of the cytostasis by a test substance, namely, cytotoxicity, for example, RICC (Relative increase in cell count) can be calculated according to the following formula based on the number of cells to be measured at the start of treatment with the test substance and at the completion of culture:

$$R I C C ( \% ) = \frac{( \begin{matrix}cell number at completion of culture in test substance \text{-} \\ treated group - cell number at start of treatment\end{matrix} )}{( \begin{matrix}cell number at completion of culture in negative \\ control group - cell number at start of treatment\end{matrix} )} \times 100$$

Alternatively, RCC (Relative cell count) may be calculated according to the following formula based on the number of cells to be measured at the start of treatment with the test substance and at the completion of culture:

$$R C C ( \% ) = \frac{( \begin{matrix}cell num ber at completion of culture in \\ test substance \text{-} treated group\end{matrix} )}{( \begin{matrix}cell number at completion of culture in \\ negative control group\end{matrix} )} \times 100$$

When chromosomal aberration inducibility is evaluated using RICC, for example, for respective test substances, the case when a 5% significant increase is not found in frequency of chromosomal aberration at all treatment doses of RICC of not less than 40% can be judged as negative, and the case when a 5% significant increase is found in frequency of chromosomal aberration at a treatment dose of RICC of not less than 40% can be judged as positive. In this case, a treatment dose of RICC of less than 40% may be excluded from the determination of the chromosomal aberration inducibility because an increase in the frequency of chromosomal aberration with low biological significance is likely to be found due to cytotoxicity. Alternatively, for respective test substances, the case when a 5% significant increase is not found in frequency of chromosomal aberration at all treatment doses of RICC of not less than 30% can be judged as negative, and the case when a 5% significant increase is found in frequency chromosomal aberration at a treatment dose of RICC of not less than 30% can be judged as positive. In this case, a treatment dose of RICC of less than 30% may be excluded from the determination of the chromosomal aberration inducibility because an increase in the frequency of chromosomal aberration with low biological significance is likely to be found due to cytotoxicity. These methods are examples of an evaluation method of chromosomal aberration inducibility, and the method is not limited thereto.

When chromosomal aberration inducibility is evaluated using RCC, for example, for respective test substances, the case when a 5% significant increase is not found in frequency of chromosomal aberration at all treatment doses of RCC of not less than 30% can be judged as negative, and the case when a 5% significant increase is found in frequency of chromosomal aberration at a treatment dose of RCC of not less than 30% can be judged as positive. In this case, a treatment dose of RCC of less than 30% may be excluded from the determination of the chromosomal aberration inducibility because an increase in the frequency of chromosomal aberration with low biological significance is likely to be found due to cytotoxicity.

### (C)<Detection of Chromosomal Aberration by Evaluation of DNA Damage>

In the present invention, the procedure of an in vitro comet assay can be utilized for detection of chromosomal aberration by “evaluation of DNA damage”. For example, a method including

(C1) a step of applying proliferation stimulation to human pluripotent stem cell-derived T lymphocyte by using the aforementioned anti-CD3 antibody,
(C2) a step of adding a test substance or a medium to be used as a negative control to the human pluripotent stem cell-derived T lymphocyte applied with the proliferation stimulation to perform a test substance treatment,
(C3) a step of recovering the cells after the treatment, embedding them in a gel, fixing them on a slide glass, treating the cells on the slide glass with a lysis buffer, removing cellular membrane and nuclear envelope to expose DNA, further exposing the DNA under strong alkali conditions to release DNA fragments etc. generated by DNA damage, and electrophoresing nuclear DNA in the gel to prepare a specimen for comet assay observation, and
(C4) a step of staining nucleic acid of the comet specimen, observing the cells under a microscope provided with an optical system compatible with the staining method to measure staining brightness, calculating an index value such as % tail DNA and so on, and comparing the index value of the test substance treatment group with that of the negative control group, i.e., a vehicle treatment group (untreated group) to evaluate DNA damage inducibility of the test substance, namely, inducibility of abnormality in the DNA constituting the chromosome can be mentioned.

Alternatively, a method including, after the above-mentioned step (C2),

(C′3) a step of fixing the cells after the treatment, and immunostaining a DNA double strand cleavage marker, and
(C′4) a step of comparing the immunostaining result with that of the negative control group, i.e., a vehicle treatment group (untreated group) to evaluate DNA damage inducibility of the test substance, namely, inducibility of abnormality in the DNA constituting the chromosome can be mentioned.

In step (C′3), the cells after the treatment may be, for example, fixed on a slide glass or fixed on a multiwall plate.

In step (C′4), the results of immunostaining may be confirmed under a fluorescence microscope, or an image analyzer such as imaging cytometer and so on may also be used. Alternatively, fixed and immunostained cell suspension may be analyzed using a cell analyzer in a flow path system such as a flow cytometer and so on.

Step (C1) can be performed similarly to the above-mentioned “detection method of the present invention”.

### (C2) Step of Performing Test Substance Treatment

The treatment period of the test substance is not particularly limited, and 1-6 hr can be typically mentioned. Most of the DNA damages detected by an in vitro comet assay are quickly repaired by the homeostatic function in the body. Thus, generally, a long-time treatment does not contribute to the improvement of sensitivity. A metabolic activation factor may be added at the time of test substance treatment to detect a test substance that induces DNA damage through metabolic activation by metabolic enzymes. Examples of the widely-used metabolic activation factor include, but are not limited to, S9mix obtained by adding coenzyme etc. to S9 which is derived from a rat liver homogenate. The addition concentration of the S9mix is not particularly limited, but is preferably used at a concentration that does not show significant toxicity or DNA damage to the cells. The concentration is preferably 1-10% (v/v).

### (C3) Step of Preparing Comet Specimen

As a gel for embedding cells, low melting point agarose gel is widely used, but the gel is not limited thereto. The composition of the lysis buffer used for removing cellular membrane and nuclear envelope of the cell is not particularly limited. A mixed buffer of surfactant and high concentration salt is widely used, and, for example, mixed buffers of Triton X-100, sodium chloride, ethylenediaminetetraacetic acid (EDTA), trihydroxymethylaminomethane (Tris), dimethyl sulfoxide (DMSO) can be mentioned. The conditions under which DNA of cell nucleus is exposed to strong alkali conditions are not particularly limited. Generally, not less than pH 13 is preferable, and an aqueous sodium hydroxide solution is widely used.

### (C4) Step of Performing Evaluation of Abnormality Inducibility

For the staining of nucleic acid in a comet specimen, SYBR (registered trade mark) Gold is widely used. However, the staining reagent is not limited thereto and various known nucleic acid staining reagents can be utilized. For the purpose of identifying DNA fragmentation due to cytotoxicity, a known apoptosis evaluation method such as measurement of caspase activity and so on can also be combined. While the number of cells to be observation object is not particularly limited, not less than 50 cells per one condition are preferable and not less than 100 cells per one condition are more preferable, to ensure statistical accuracy. The observation step of the stained comet specimen can also be automated by using an image analyzer. As an application example of the image analyzer, “Toxicol In Vitro 2009, 23(8), p 1570-1575” and so on have been reported but it is not limited thereto.

In addition, to facilitate testing of many samples, it is also possible to prepare a comet specimen on the surface of a culture vessel such as a 96-well plate without collecting the cells. Using the aforementioned image analyzer, high-throughput automatic analysis can be performed. When determining DNA damage inducibility by a test substance, various statistical tests can be performed between the index value such as % tail DNA and so on in the test substance group and the index value in the negative control group. It can also be determined by the comparison of the range of the past achievements of the index value in the negative control group (historical control data) with the index value in the test substance group. While the statistical test method to be used is not particularly limited, multiple comparison by the Dunnett's test and a test of increase tendency by Cochran-Armitage tendency test are frequently used. In practicing an in vitro comet assay, for the purpose of analyzing the DNA damage induction mechanism in detail, DNA repair enzymes such as endonuclease-III (Endo III), formamidopyrimidine-DNA glycosylase (FPG), 8-oxoguanine DNA glycosylase (OGG1) and so on may be used. As a modified comet assay, “Mutat Res 2011, 726(2), p 242-250” and so on have been reported but it is not limited thereto.

The % tail DNA can be calculated as a percentage of the fluorescence brightness of the tail to the fluorescence brightness of the whole cell nucleus. Median % tail DNA of total observed nuclei (50 or more) is taken per specimen, mean of median of % tail DNA obtained from two pieces of specimen for one condition is taken as the measured value of % tail DNA under this condition, whereby an index of DNA damage by the test substance is obtained. In addition, a bell-shaped nucleus (hedgehog) recognized as a sign of apoptosis due to cytotoxicity of the test substance may be excluded from the calculation targets of % tail DNA.

For example, the presence or absence of a significant increase with significance probability (two-sided, 5% or 1%) may be tested by the Dunnett's test between the % tail DNA in a test substance treatment group and the % tail DNA in the corresponding negative control group.

As an index of the cytostasis by a test substance, namely, cytotoxicity, the above-mentioned RICC (Relative increase in cell count) can be calculated based on the number of cells at the start of treatment with the test substance and at the completion of culture. When chromosomal aberration inducibility is evaluated using RICC, for example, for respective test substances, the case when a 5% significant increase is not found in % tail DNA at all treatment doses of RICC of not less than 40% can be judged as negative, and the case when a 5% significant increase is found in % tail DNA at a treatment dose of RICC of not less than 40% can be judged as positive. A treatment dose of RICC of less than 40% may be excluded from the determination of the DNA damage because an increase in the % tail DNA with low biological significance is likely to be found due to cytotoxicity. The method is an example of an evaluation method of chromosomal aberration inducibility, and the method is not limited thereto.

Alternatively, as an index of the cytostasis by a test substance, namely, cytotoxicity, the above-mentioned RCC (Relative cell count) can be calculated based on the number of cells at the start of treatment with the test substance and at the completion of culture. When chromosomal aberration inducibility is evaluated using RCC, for example, for respective test substances, the case when a 5% significant increase is not found in % tail DNA at all treatment doses of RCC of not less than 30% can be judged as negative, and the case when a 5% significant increase is found in % tail DNA at a treatment dose of RCC of not less than 30% can be judged as positive. A treatment dose of RCC of less than 30% may be excluded from the determination of the DNA damage because an increase in the % tail DNA with low biological significance is likely to be found due to cytotoxicity. The method is an example of an evaluation method of chromosomal aberration inducibility, and the method is not limited thereto.

### (C′3) Step of Performing Immunostaining

Immunostaining can be performed by a method known per se. While DNA double strand cleavage marker is not particularly limited as long as it can detect double strand cleavage of DNA, it is preferable to use γH2AX as the marker. Therefore, for example, immunostaining can be performed using anti-γH2AX antibody as the primary antibody and HRP-conjugated anti-rabbit/mouse IgG antibody as the secondary antibody.

### (C′4) Step of Performing Evaluation of Abnormality Inducibility

While the number of cells to be observation object is not particularly limited, not less than 50 cells per one condition are preferable and not less than 100 cells per one condition are more preferable, to ensure statistical accuracy. The observation step of the immunostained specimen can also be automated by using an image analyzer.

A kit containing the aforementioned anti-CD3 antibody and pluripotent stem cell-derived T lymphocyte is utilizable for evaluation of potential to induce chromosomal aberration possessed by environmental factors such as radiation and various chemical substances, and so on, and screening of chemical substances that do not induce chromosomal aberration by applying detection or test method of the present invention. A preferable pluripotent stem cell-derived T lymphocyte is, for example, human-derived T lymphocyte. The kit may contain, where necessary, other equipment, reagents and so on as well as the aforementioned anti-CD3 antibody and pluripotent stem cell-derived T lymphocyte. Examples of other equipment and reagents include a nucleic acid staining agent, a cytokinesis inhibitor, a metabolic activation reagent such as S9mix and so on, a medium used for recovery and culture of frozen cells, a cell culture vessel such as 96 well plate and so on, and so on. In addition, to mainly mimic the in vivo environment more precisely, a culture vessel (organ on-chip) in which pluripotent stem cell-derived T lymphocytes, hepatocytes, and so on are disposed on a microfluidic device may also be a component of the kit.

The present invention is explained in more detail in the following by referring to Examples, which are not to be construed as limitative. As the culture conditions in the present Example, the medium is in a neutral range, CO₂ concentration is 5%, and temperature is 37° C.

### EXAMPLES

### Example 1 Division Kinetics of Human iPS Cell-Derived T Lymphocyte Stimulated with Anti-CD3 Antibody

### (1) Human iPS Cell-Derived T Lymphocyte

Human iPS cell lines of “2” and “12” established in Kaneko Laboratory, Center for iPS Cell Research and Application, Kyoto University, from human peripheral blood T lymphocytes according to the method described in “Nishimura, T. et al. Cell Stem Cell 2013, 12, p 114-126” were induced to differentiate into T lymphocytes which are CD8SP (Single Positive) according to the method described in “Nishimura, T. et al. Cell Stem Cell 2013, 12, p 114-126”.

The obtained CDBSP T lymphocytes (the cells are hereinafter indicated as redifferentiated T lymphocyte) were subjected to proliferation stimulation under 6 conditions shown in (2). After proliferation stimulation, the cytokinesis inhibitor CytoB was added to prepare specimens (specimen b-g). The division kinetics thereof were evaluated by measuring the distribution of the number of nucleus per cell. As the medium to which a proliferation stimulation factor is added, L-glutamine-containing RPMI-1640 medium (Wako) supplemented with 10% human serum AB (Nova Biologics), 1% Penicillin-Streptomycin (nacalai tesque), 10 ng/mL IL-7 (Wako), and 10 ng/mL IL-15 (R&D SYSTEMS) (hereinafter to be indicated as Rh medium) was used.

As a comparison control, the cells subjected to maintenance culture alone in Rh medium without proliferation stimulation were similarly evaluated for the division kinetics.

### (2) Proliferation Stimulation Conditions

### <Maintenance Culture Conditions>

Redifferentiated T lymphocytes derived from human iPS cell line “2” described in (1) were suspended in Rh medium at a cell density of 1.1×10⁶ cells/mL, and seeded by 100 μL on a 96 well plate (Techno Plastic Products AG). After culture for about 46 hr, the culture supernatant (5 μL) was substituted with Rh medium (5 μL) supplemented with 120 μg/mL CytoB (Wako) (final concentration of CytoB: 6 μg/mL). After the addition of CytoB and further culture for about 30 hr, a specimen was prepared according to the process described in (3). The specimen is hereinafter indicated as specimen a.

### <Conditions of Proliferation Stimulation by Magnetic Bead-Bound Anti-CD3 Antibody>

As a magnetic bead-bound anti-CD3 antibody, Dynabeads (registered trade mark) Human T-Activator CD3/CD28 (VERITAS) was used. Dynabeads (registered trade mark) Human T-Activator CD3/CD28 in a 3-fold amount were added to Redifferentiated T lymphocytes (1.1×10⁵ cells) derived from human iPS cell line “2” described in (1), and suspended in Rh medium (hereinafter to be indicated as Rh-2 medium) (100 μL) supplemented with 100 U/mL IL-2 (Wako). The suspension was shaken at about 6 rpm at room temperature for about 1 hr, and the total amount was seeded on a 96 well plate (Techno Plastic Products AG) (final cell density: 1.1×10⁶ cells/mL). After culture for about 45 hr, the culture supernatant (5 μL) was substituted with Rh-2 medium (5 μL) supplemented with 120 μg/mL CytoB (final concentration of CytoB: 6 μg/mL). After the addition of CytoB and further culture for about 30 hr, a specimen was prepared according to the process described in (3). That is, the culture period after anti-CD3 antibody addition was set to about 75 hr. The specimen is hereinafter indicated as specimen b.

### <Conditions of Proliferation Stimulation by Anti-CD3 Antibody-Bound Plate-1>

As the anti-CD3 antibody-bound plate, BioCoat™ T cells activated, anti-human CD3 96 well flat bottom assay plate (Corning) was used.

Redifferentiated T lymphocytes derived from human iPS cell line “2” described in (1) were suspended in Rh-2 medium at a cell density of 5.0×10⁵ cells/mL, and seeded by 160 μL each in 3 wells of the anti-CD3 antibody-bound plate (final cell density: 5.0×10⁵ cells/mL). After culture for about 43 hr, the culture supernatant (16 μL) was substituted with Rh-2 medium (16 μL) supplemented with 60 μg/mL CytoB (final concentration of CytoB: 6 μg/mL). After further culture for about 24 hr, about 29 hr, or about 48 hr, a specimen was prepared according to the process described in (3). That is, the culture period after anti-CD3 antibody addition was set to about 67 hr, about 72 hr, about 91 hr, respectively. In the following, the specimen with culture period of about 24 hr after addition of CytoB is indicated as specimen c, the specimen with culture period of about 29 hr after addition of CytoB is indicated as specimen d, and the specimen with culture period of about 48 hr after addition of CytoB is indicated as specimen e.

### <Proliferation Stimulation Conditions by Anti-CD3 Antibody-Bound Plate-2>

As an anti-CD3 antibody-bound plate, a 96 well plate (nunc) was used after coating with an anti-CD3 antibody when in use. 0.5 μg/mL or 1.0 μg/mL Anti-Human CD3 Functional Grade Purified (eBioscience) (50 μL) was added to the 96 well plate, and the plate was stood at room temperature for 7 hr to coat the plate surface with the antibody. Each well was washed twice with 100 μL of phosphate-buffered saline (PBS, Nissui Pharmaceutical Co., Ltd.), and PBS was substituted with 40 μL of Rh-2 medium.

Redifferentiated T lymphocytes derived from human iPS cell line “12” described in (1) were suspended in Rh-2 medium at a cell density of 2.1×10⁶ cells/mL and seeded by 40 μL in each well (final cell density: 1.0×10⁶ cells/mL). After about 16 hr of culture, each well was pipetted to recover the total amount of the cell suspension. That is, the culture period after addition of the anti-CD3 antibody (period after anti-CD3 antibody addition (start of proliferation stimulation) to removal of anti-CD3 antibody from culture system) was set to about 16 hr. The cell suspension was diluted with Rh-2 medium to a cell density of 5.6×10⁵ cells/mL and added to a 96 well plate not coated with the antibody. Furthermore, Rh-2 medium (70 μL) was added to each well to the total amount of 150 μL and culture was continued. After about 3 hr, the culture supernatant (15 μL) was substituted with Rh-2 medium (15 μL) supplemented with 60 μg/mL CytoB (final concentration of CytoB: 6 μg/mL). After culture for about 33 hr after the addition of CytoB, a specimen was prepared according to the process described in (3). In the following, a specimen with an antibody concentration of 0.5 μg/mL on coating with the anti-CD3 antibody is indicated as specimen f, and a specimen with 1.0 μg/mL is indicated as specimen g.

The proliferation stimulation conditions of the above-mentioned specimens a-g are shown in Table 1.

### (3) Specimen Preparation Method and Division Kinetics Evaluation Method

The plate after completion of culture in (2) was centrifuged (1500 rpm, 2 min), and the supernatant was substituted with PBS. The operation was repeated twice, and the cell suspension was sufficiently washed. After washing, the plate was centrifuged again (1500 rpm, 2 min), the supernatant was removed, 75 mM KCl (Wako) heated to 37° C. was added and a hypotonic treatment was performed for 5 min. To the cell suspension after the hypotonicity was added 1/5 volume of a fixation solution (methanol (nacalai tesque):glacial acetic acid (nacalai tesque)=3:1), and the cells were semi-fixed and centrifuged (4° C., 1500 rpm, 5 min). After centrifugation, the supernatant was substituted with a fresh fixation solution, and the cells were fixed and centrifuged (4° C., 1500 rpm, 5 min). The operation was repeated twice, and the cells were sufficiently fixed. The supernatant was substituted with methanol, the cell suspension was recovered in an eppendorf tube by sufficient pipetting and centrifuged (4° C., 10000 rpm, 5 min). The supernatant was removed, and the residue was resuspended in methanol to a cell density at which the suspension became slightly cloudy and the suspension was added dropwise on a slide glass and air dried to give a specimen. The prepared specimen was stained with 40 μg/mL Acridine Orange (Wako) solution, and the cells were observed under a fluorescence microscope using a wide-band Blue excitation filter. The cells were classified into mononucleated cells having one main nucleus, binucleated cells having two main nuclei, and multinucleated cells having not less than three main nuclei, in the cytoplasm, and counted. Not less than 1000 redifferentiated T lymphocytes were observed per specimen and mononucleated cell number, binucleated cell number and multinucleated cell number were measured.

As an index of division kinetics, CBPI (Cytokinesis-block proliferation index) was calculated according to the following formula. Since CBPI depends on the treatment time of CytoB, there is no clear consensus on the standard value to secure a good division state. However, generally, the cells are sufficiently practical as a detection and test system if it is 1.5 or more.

$$CBPI = \frac{( \begin{matrix}mononucleated cell number + 2 \times binucleated cell number + \\ 3 \times multinucleated cell number\end{matrix} )}{total observed cell number}$$

### (4) Results

The results are shown in Table 2.

Specimen a without proliferation stimulation showed CBPI 1.09. On the other hand, specimens b-g showed CBPI 1.53-1.92. Therefore, it was demonstrated that redifferentiated T lymphocyte scarcely divides without a proliferation stimulation factor; however, cell division of a level practical sufficiently as a detection, testing system of chromosomal aberration is induced under any growth stimulation condition using an anti-CD3 antibody.

TABLE 1
List of proliferation stimulation conditions used

| | proliferation stimulation | CytoB |
|---|---|---|
| specimen | conditions | treatment time |
| a | no proliferation stimulation | 30 hours |
| b | Dynabeads (registered trade mark) | 30 hours |
| | Human T-Activator CD3/CD28 | |
| c | BioCoat ™ anti-human CD3 96 well | 24 hours |
| | plate | |
| d | BioCoat ™ anti-human CD3 96 well | 29 hours |
| | plate | |
| e | BioCoat ™ anti-human CD3 96 well | 48 hours |
| | plate | |
| f | 96 well plate coated with 0.5 μg/mL | 33 hours |
| | anti-CD3 antibody when in use | |
| g | 96 well plate coated with 1.0 μg/mL | 33 hours |
| | anti-CD3 antibody when in use | |

TABLE 2
Division kinetics of human iPS cell-derived T lymphocyte
by stimulation with anti-human CD3 antibody

| | | mono- | bi- | multi- | |
|---|---|---|---|---|---|
| | observed | nucleated | nucleated | nucleated | |
| | cell | cell | cell | cell | |
| specimen | number | number | number | number | CBPI |
| a | 1000 | 913 | 83 | 4 | 1.09 |
| b | 1000 | 351 | 520 | 129 | 1.78 |
| c | 1000 | 481 | 511 | 8 | 1.53 |
| d | 1000 | 392 | 560 | 48 | 1.66 |
| e | 1000 | 271 | 539 | 190 | 1.92 |
| f | 1100 | 392 | 660 | 48 | 1.69 |
| g | 1000 | 394 | 568 | 38 | 1.64 |

### Example 2 In Vitro Micronucleus Test Using Human iPS Cell-Derived T Lymphocyte

### (1) Human iPS Cell-Derived T Lymphocyte

As human iPS cell-derived T lymphocyte, redifferentiated T lymphocyte derived from human iPS cell line “12” described in Example 1(1) was used.

### (2) Test Substance

As a test substance, 5 compounds of mitomycin C (MMC, CAS No. 50-07-7, Kyowa Hakko Kirin Co., Ltd.), cytosine arabinoside (AraC, CAS No. 147-94-4, nacalai tesque), ethyl methanesulfonate (EMS, CAS No. 62-50-0, nacalai tesque), bleomycin sulfate (BLM, CAS No. 9041-93-4, LKT laboratories) and 1-methyl-3-nitro-1-nitrosoguanidine (MNNG, CAS No. 70-25-7, nacalai tesque) were used as compounds that induce micronucleus resulted from chromosomal structural aberration, 2 compounds of colchicine (COL, CAS No. 64-86-8, Wako) and vinblastine sulfate (VBL, CAS No. 143-67-9, Wako) were used as compounds that induce micronucleus resulted from chromosomal numerical aberration, cyclophosphamide monohydrate (CPA, CAS No. 6055-19-2, nacalai tesque) was used as a compound that induces micronucleus after metabolic activation by metabolic enzymes in vivo, and D(−)-mannitol (MAN, CAS No. 69-65-8, Wako) was used as a compound that does not induce micronucleus. All the 9 compounds above have reactivity (chromosomal aberration inducibility) sufficiently evaluated in many known documents and so on (e.g., Mutat Res, 2006, 607, p 37-60, Mutagenesis, 2011, 26(6), p 763-770 and so on). A test substance was used at the treatment dose (final concentration in medium) described in Table 3. For the treatment, a test substance was dissolved in physiological saline (prepared by dissolving sodium chloride (Wako) in ultrapure water, and sterilizing in autoclave) or DMSO (Dojin chemical) at a 100-fold concentration of the final concentration in the medium for addition at 1% (v/v). CPA was treated in the presence of rat liver homogenate S9 as a metabolic activation system. Treatment with a test substance was performed according to the process described in (4).

### (3) Proliferation Stimulation Method

For proliferation stimulation of redifferentiated T lymphocytes, a 12 well plate was used after coating with an anti-CD3 antibody when in use. 1.0 μg/mL Anti-Human CD3 Functional Grade Purified (1 mL) was added to a 12 well plate (nunc), and the plate was stood at room temperature for about 6 hr to coat the plate surface with the antibody. Each well was washed twice with 1 mL of PBS, redifferentiated T lymphocytes derived from human iPS cell line “12” described in (1) were suspended in Rh-2 medium at a cell density of 1.0×10⁶ cells/mL and seeded in each well (final cell density: 1.0×10⁶ cells/mL). After about 17 hr of culture, the total amount of the cell suspension was recovered by pipetting. That is, the culture period after addition of the anti-CD3 antibody (period after anti-CD3 antibody addition (start of proliferation stimulation) to removal of anti-CD3 antibody from culture system) was set to about 17 hr. The cell suspension was diluted with Rh-2 medium to a cell density of 4.0×10⁵ cells/mL and reseeded in a 96 well plate not coated with the antibody at 150 μL per well, and culture was continued.

### (4) Test Substance Treatment Method

Treatments with 8 compounds other than CPA were performed according to the method described below. That is, after about 23 hr (culture period from removal of anti-CD3 antibody to addition of test substance) from the reseeding in (3), the culture supernatant (15 μL) was substituted with Rh-2 medium (15 μL) supplemented with 60 μg/mL CytoB (final concentration of CytoB: 6 μg/mL). Furthermore, a test substance (1.5 μL) dissolved in physiological saline or DMSO at a 100-fold concentration of the final concentration described in Table 3 was added and the cells were treated for 33 hr.

Different from a test substance treatment group, DMSO (1.5 μL) was added and a similar treatment was performed for 33 hr to prepare a negative control group.

CPA treatment was performed according to the method described below. That is, after about 21 hr from reseeding in (3) (culture period from removal of anti-CD3 antibody to addition of test substance), Rh-2 medium (30 μL) supplemented with 6% (v/v) S9 (rat liver 9000×g supernatant fraction induced with phenobarbital and 5,6-benzoflavone, ORIENTAL YEAST), 0.8 mM HEPES (2-[4-(2-hydroxyethyl)-1-piperazinyl]ethanesulfonic acid, nacalai tesque), 1 mM MgCl₂ (Wako), 6.6 mM KCl, 1 mM Glucose-6-phosphate (ORIENTAL YEAST), 0.8 mM NADPH (ORIENTAL YEAST) was added to the well (final concentration of S9:1% (v/v)). Furthermore, CPA solution (1.8 μL) obtained by dissolving in physiological saline at a 100-fold concentration of the final concentration described in Table 3 was added and treatment was performed for 3 hr.

Different from the CPA treatment group, physiological saline (1.8 μL) was added and a similar treatment was performed for 33 hr to give a negative control group.

After the treatment for 3 hr, the plate was centrifuged (1500 rpm, 3 min), and the supernatant was substituted with Rh-2. The operation was repeated 3 times, and the cell suspension was sufficiently washed. After washing, the plate was centrifuged again (1500 rpm, 3 min), the supernatant was removed, and Rh-2 medium was added to make the total amount 135 μL. Furthermore, Rh-2 medium (15 μL) supplemented with 60 Kg/mL CytoB was added (final concentration of CytoB: 6 μg/mL), and the mixture was cultured for 33 hr.

### (5) Specimen Preparation Method and Micronucleus Inducibility Evaluation Method

The plate after treatment and completion of culture in (4) was centrifuged (1500 rpm, 3 min), and the supernatant was substituted with PBS. The operation was repeated twice, and the cell suspension was sufficiently washed. After washing, the plate was centrifuged again (1500 rpm, 3 min), and the supernatant was substituted with Accutase (Innovative cell technologies), sufficiently pipetted to loosen the aggregate of T lymphocytes.

The plate was further centrifuged (1500 rpm, 3 min), the supernatant was removed, 75 mM KCl heated to 37° C. was added and a hypotonic treatment was performed for 5 min. To the cell suspension after the hypotonicity was added 1/5 volume of a fixation solution (methanol:glacial acetic acid=3:1), and the cells were semi-fixed and centrifuged (4° C., 1500 rpm, 3 min). After centrifugation, the supernatant was substituted with a fresh fixation solution, and the cells were fixed and centrifuged (4° C., 1500 rpm, 3 min). The operation was repeated twice, and the cells were sufficiently fixed. The supernatant was substituted with methanol, the mixture was centrifuged (4° C., 1500 rpm, 3 min) and the cells were suspended in a small amount of methanol. The suspension was added dropwise on a slide glass and air dried to give a specimen.

In AraC 0.5 μg/mL and 1.0 μg/mL treatment groups, VBL 25 ng/mL and 50 ng/mL treatment groups, the number of the cells was markedly small due to remarkable cytotoxicity, and the specimen could not be prepared.

The prepared specimen was stained with 40 g/mL Acridine Orange solution, and the cells were observed under a fluorescence microscope using a wide-band Blue excitation filter. Not less than 500 cells were observed per condition and mononucleated cell number, binucleated cell number and multinucleated cell number were measured. In addition, not less than 500 binucleated cells were observed per 1 condition and the number of binucleated cells having micronucleus was measured. For conditions under which 500 cells or binucleated cells cannot be observed due to cytotoxicity of the test substance, all cells or binucleated cells on a slide glass were observed. As the determination criteria of micronucleus, a circular nucleus-like structure with a radius of ⅓ or less of that of the main nucleus and having fluorescence intensity equal to that of the main nucleus is regarded as micronucleus. Binucleated cell with 4 or more micronuclei, cell with large differences in size and fluorescence intensity among multiple main nuclei, and cell with markedly irregular shape of main nucleus or micronucleus were removed from the observation target since they are highly likely in a non-physiological state such as cell death and so on.

As an index of micronucleus inducibility by a test substance, frequency of micronucleus was obtained by dividing the number of binucleated cells with micronucleus by the total number of binucleated cells. In addition, the Fisher's exact test was performed between the frequency of micronucleus in a test substance treatment group and the frequency of micronucleus in the corresponding negative control group, and the presence or absence of a significant increase with significance probability (two-sided, 5% or 1%) was tested.

As an index of the cell division inhibitory action of a test substance, namely, cytotoxicity, CBPI was calculated as in Example 1(3) and RI (Replication index) was calculated:

$$R I ( \% ) = \frac{( CBPI in test substance \text{-} treated group - 1 )}{( CBPI in negative control group - 1 )} \times 100$$

For respective test substances, the case when a 5% significant increase was not found in frequency of micronucleus at all treatment doses of RICC of not less than 40% was judged as negative, and the case when a 5% significant increase was found in frequency of micronucleus at a treatment dose of RICC of not less than 40% was judged as positive. A treatment dose of RICC of less than 40% was excluded from the determination of the micronucleus inducibility because an increase in the frequency of micronucleus with low biological significance was likely to be found due to cytotoxicity.

### (6) Results

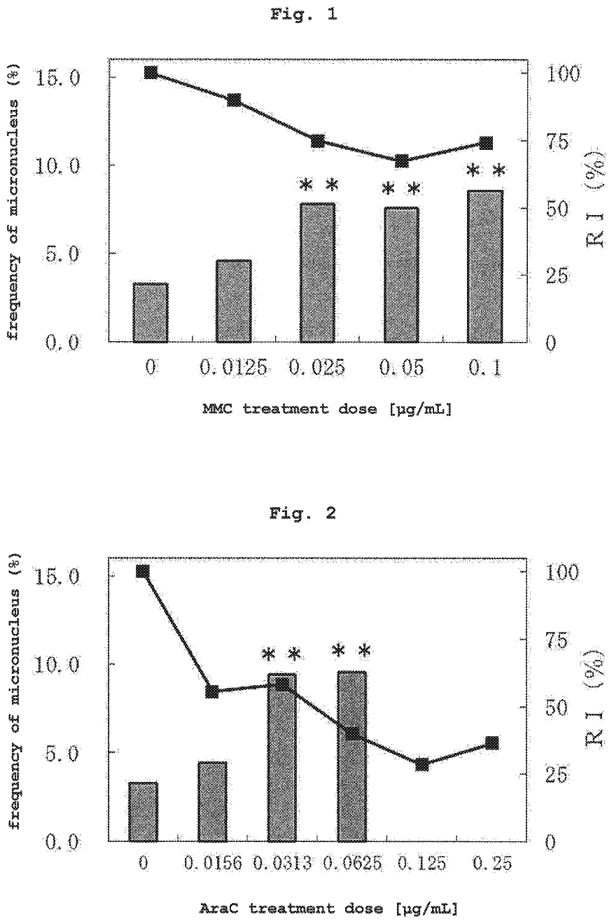

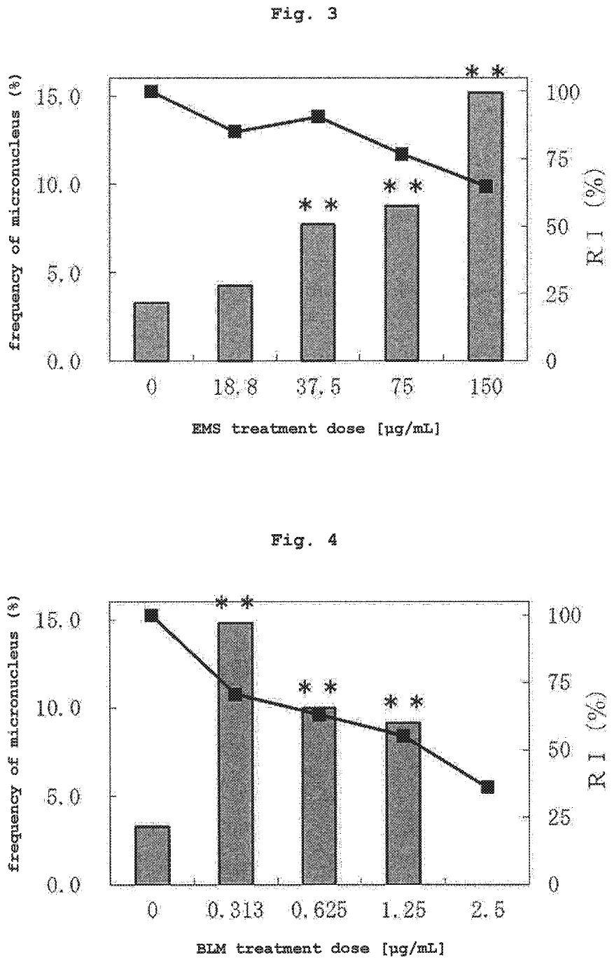

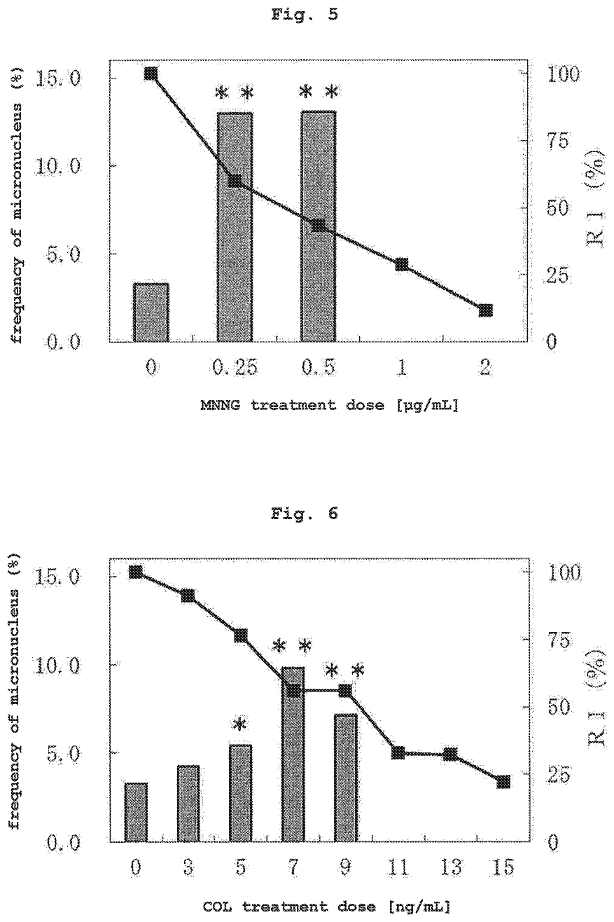

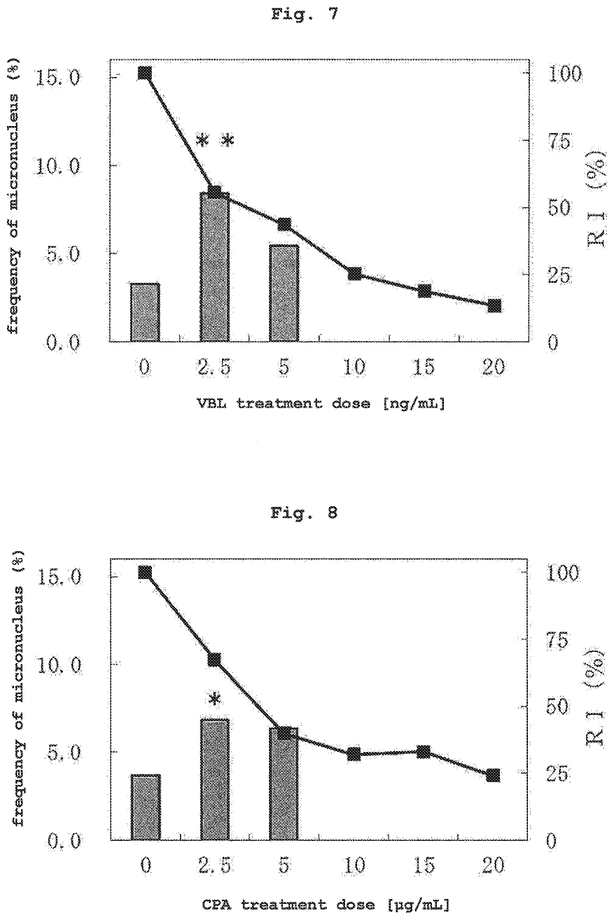

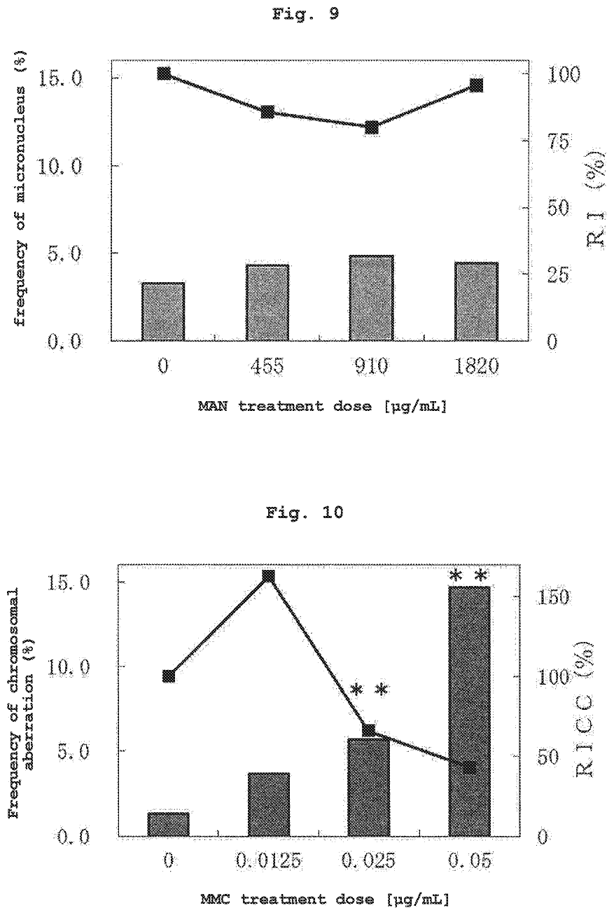

The judgment results obtained in this Example for the nine compounds as test compounds and the judgment results expected from known information (Kirkland, D. et al. Mutation Research 2016, 795, 7-30; Lasne, C. et al. Mutation Research 1984, 130(4), 273-282; Aardema M J. et al. Mutation Research 2006, 607(1), 61-87) are shown in Table 4. In addition, the dose response relationships of respective test substances are shown in FIG. 1-FIG. 9.

As shown in Table 4 and FIG. 1-FIG. 9, the responsiveness expected from known information could be correctly detected for all of the nine compounds as test compounds including a structural aberration inducible compound, a numerical aberration inducible compound, a structural aberration inducible compound requiring metabolic activation, and a micronucleus non-inducible compound. That is, it was demonstrated that the in vitro micronucleus test using human iPS cell-derived T lymphocyte can be practically used for the evaluation of micronucleus inducibility of a test substance.

TABLE 3
List of test substance treatment dose
used for in vitro micronucleus test

| test | | |
|---|---|---|
| substance | solvent | treatment dose [μg/mL] |
| MMC | physiological saline | 0.0125, 0.025, 0.05, 0.1 |
| AraC | DMSO | 0.0156, 0.0313, 0.0625, |
| | | 0.125, 0.25, 0.5, 1 |
| EMS | DMSO | 18.8, 37.5, 75, 150 |
| BLM | DMSO | 0.313, 0.625, 1.25, 2.5 |
| MNNG | DMSO | 0.25, 0.5, 1, 2 |
| COL | physiological saline | 0.003, 0.005, 0.007, 0.009, |
| | | 0.011, 0.013, 0.015 |
| VBL | DMSO | 0.0025, 0.005, 0.01, 0.015, |
| | | 0.02, 0.025, 0.05 |
| CPA | physiological saline | 2, 5, 10, 15, 20 |
| MAN | physiological saline | 455, 910, 1820 |

TABLE 4
Outline of results of in vitro micronucleus test
using human iPS cell-derived T lymphocyte

| test | judgment result obtained | judgment result expected |
|---|---|---|
| substance | in this Example | from known information |
| MMC | positive | positive |
| AraC | positive | positive |
| EMS | positive | positive |
| BLM | positive | positive |
| MNNG | positive | positive |
| COL | positive | positive |
| VBL | positive | positive |
| CPA | positive | positive |
| MAN | negative | negative |

### Example 3 In Vitro Chromosomal Aberration Test Using Human iPS Cell-Derived T Lymphocyte

### (1) Human iPS Cell-Derived T Lymphocyte

As human iPS cell-derived T lymphocyte, a redifferentiated cell for which a human iPS cell line established in Kaneko Laboratory, Center for iPS Cell Research and Application, Kyoto University, from human peripheral blood T lymphocytes according to the method described in “Nishimura, T. et al. Cell Stem Cell 2013, 12, p 114-126” were induced to differentiate according to the method described in “Nishimura, T. et al. Cell Stem Cell 2013, 12, p 114-126” is used.

### (2) Test Substance

As a test substance, EMS is used as a compound that induces chromosomal structural aberration and MAN is used as a compound that does not induce chromosomal structural aberration. A test substance is used at the treatment dose (final concentration in medium) described in Table 5. For the treatment, a test substance is dissolved in physiological saline at a 100-fold concentration of the final concentration in the medium for addition at 1% (v/v). Treatment with a test substance is performed according to the process described in (4).

### (3) Proliferation Stimulation Method

For proliferation stimulation of redifferentiated T lymphocytes, a 12 well plate is used after coating with an anti-CD3 antibody when in use. 1.0 μg/mL Anti-Human CD3 Functional Grade Purified (1 mL) is added to a 12 well plate, and the plate is stood at room temperature for about 6 hr to coat the plate surface with the antibody. Each well is washed twice with 1 mL of PBS, redifferentiated T lymphocytes derived from human iPS cell line described in (1) are suspended in Rh-2 medium at a cell density of 1.0×10⁶ cells/mL and added to each well. After about 17 hr of culture, the total amount of the cell suspension is recovered by pipetting. The cell suspension is diluted with Rh-2 medium to a cell density of 4.0×10⁵ cells/mL and reseeded in a 12 well plate not coated with the antibody at 1 mL per well, and culture is continued.

### (4) Test Substance Treatment Method

Treatments with EMS and MAN are performed according to the method described below. That is, after about 23 hr from the reseeding in (3), a test substance (10 μL) dissolved in physiological saline at a 100-fold concentration of the final concentration described in Table 5 is further added and the cells are treated for 33 hr. Different from a test substance treatment group, physiological saline (10 μL) is added and a similar treatment is performed for 33 hr to prepare a negative control group. About 2 hr before completion of the treatment, KaryoMAX (registered trade mark) Colcemid™ Solution in HBSS (Life technologies) (10 μL) is added to the well (final concentration in medium: 0.1 μg/mL) to discontinue the cell cycle, and cells in mitotic metaphase are enriched.

At the time of start of the test substance treatment, a part of the cell suspension of the negative control group is separated and the number of the cells is measured using a counting chamber.

### (5) Specimen Preparation Method and Evaluation Method of Chromosomal Aberration Inducibility

A part of the cell suspension after the treatment and completion of culture in (4) was separated, and the number of cells is measured using a counting chamber. A plate containing the remaining cell suspension is centrifuged (1500 rpm, 3 min), and the supernatant is substituted with PBS. The operation is repeated twice, and the cell suspension is sufficiently washed. After washing, the plate is centrifuged again (1500 rpm, 3 min), and the supernatant is substituted with Accutase (Innovative cell technologies), sufficiently pipetted to loosen the aggregate of T lymphocytes. The plate is further centrifuged

(1500 rpm, 3 min), the supernatant is removed, 75 mM KCl heated to 37° C. is added and a hypotonic treatment is performed for 5 min. To the cell suspension after the hypotonicity is added 1/5 volume of a fixation solution (methanol:glacial acetic acid=3:1), and the cells are semi-fixed and centrifuged (4° C., 1500 rpm, 3 min). After centrifugation, the supernatant is substituted with a fresh fixation solution, and the cells are fixed and centrifuged (4° C., 1500 rpm, 3 min). The operation is repeated 3 times, and the cells are sufficiently fixed and centrifuged again (4° C., 1500 rpm, 3 min). The cells are suspended in a small amount of a fixation solution. The suspension is added dropwise on a slide glass and air dried to give a specimen.

The prepared specimen is stained with Giemsa and observed under a stereomicroscope at 1000-fold magnification. The chromosomes are observed in not less than 50 cells in mitotic metaphase having well-spread chromosomes per condition to determine the presence or absence of structural aberration of the chromosome. For conditions under which 50 cells in mitotic metaphase cannot be observed due to cytotoxicity of the test substance, all cells in mitotic metaphase on a slide glass are observed.

Chromosomal abnormalities are classified into chromatid break, chromosome break, chromatid exchange, chromosome exchange, and other structural abnormalities and counted. Of the chromatid or chromosome break, when the unstained site is smaller than the width of the chromatid and does not deviate from the axis of the chromatid, the unstained site is a chromatid or chromosome gap and is not included in the chromosomal aberration.

As an index of chromosomal aberration inducibility by a test substance, frequency of chromosomal aberration is obtained by dividing the number of cells in mitotic metaphase with any chromosomal aberration by the total number of observed cells in mitotic metaphase. In addition, the Fisher's exact test is performed between the frequency of chromosomal aberration in a test substance treatment group and the frequency of chromosomal aberration in the corresponding negative control group, and the presence or absence of a significant increase with significance probability (two-sided, 5% or 1%) is tested.

As an index of the cytostasis by a test substance, namely, cytotoxicity, RICC (Relative increase in cell count) is calculated according to the following formula and based on the number of cells at the start of the test substance treatment and at the completion of culture, which are measured in (4):

$$R I C C ( \% ) = \frac{( \begin{matrix}cell number at completion of culture in test substance \text{-} \\ treated group - cell number at start of treatment\end{matrix} )}{( \begin{matrix}cell number at completion of culture in negative \\ control group - cell number at start of treatment\end{matrix} )} \times 100$$

For respective test substances, the case when a 5% significant increase is not found in frequency of chromosomal aberration at all treatment doses of RICC of not less than 40% is judged as negative, and the case when a 5% significant increase is found in frequency of chromosomal aberration at a treatment dose of RICC of not less than 40% is judged as positive.

A treatment dose of RICC of less than 40% is excluded from the determination of the chromosomal aberration inducibility because an increase in the frequency of chromosomal aberration with low biological significance is likely to be found due to cytotoxicity.

TABLE 5
List of test substance treatment dose used
for in vitro chromosomal aberration test

| test substance | treatment dose [μg/mL] |
|---|---|
| EMS | 18.8, 37.5, 75, 150 |
| MAN | 455, 910, 1820 |

### Example 4 In Vitro Comet Assay Using Human iPS Cell-Derived T Lymphocyte

### (1) Human iPS Cell-Derived T Lymphocyte

As human iPS cell-derived T lymphocyte, a redifferentiated cell for which a human iPS cell line established in Kaneko Laboratory, Center for iPS Cell Research and Application, Kyoto University, from human peripheral blood T lymphocytes according to the method described in “Nishimura, T. et al. Cell Stem Cell 2013, 12, p 114-126” were induced to differentiate according to the method described in “Nishimura, T. et al. Cell Stem Cell 2013, 12, p 114-126” is used.

### (2) Test Substance

As a test substance, EMS is used as a compound that induces DNA damage and MAN is used as a compound that does not induce DNA damage. A test substance is used at the treatment dose (final concentration in medium) described in Table 6. For the treatment, a test substance is dissolved in DMSO at a 100-fold concentration of the final concentration in the medium for addition at 1% (v/v). Treatment with a test substance is performed according to the process described in (4).

### (3) Proliferation Stimulation Method

For proliferation stimulation of redifferentiated T lymphocytes, a 12 well plate is used after coating with an anti-CD3 antibody when in use. 1.0 μg/mL Anti-Human CD3 Functional Grade Purified (1 mL) is added to a 12 well plate, and the plate is stood at room temperature for about 6 hr to coat the plate surface with the antibody. Each well is washed twice with 1 mL of PBS, redifferentiated T lymphocytes derived from human iPS cell line described in (1) are suspended in Rh-2 medium at a cell density of 1.0×10⁶ cells/mL and added to each well. After 17 hr of culture, the total amount of the cell suspension is recovered by pipetting. The cell suspension is diluted with Rh-2 medium to a cell density of 4.0×10⁵ cells/mL and reseeded in a 12 well plate not coated with the antibody at 1 mL per well, and culture is continued.

### (4) Test Substance Treatment Method

Treatments with EMS and MAN are performed according to the method described below. That is, after about 23 hr from the reseeding in (3), a solution (10 μL) obtained by dissolving a test substance in DMSO at a 100-fold concentration of the final concentration described in Table 6 is further added and the cells are treated for 4 hr. Different from a test substance treatment group, DMSO (10 μL) was added and a similar treatment was performed for 4 hr to prepare a negative control group.

Different from the samples for the preparation of comet specimens, as a sample for cytotoxicity evaluation, treatments with EMS, MAN and DMSO are similarly performed for 4 hr. As for the sample for cytotoxicity evaluation, after the treatment for 4 hr, the cells are washed with PBS and reseeded, and additionally cultured for 33 hr.

At the time of start of the test substance treatment, at completion of the treatment (at washing), and at completion of the additional culture, a part of the cell suspension is separated and the number of the cells is measured using a counting chamber.

### (5) Specimen Preparation Method and Evaluation Method of DNA Damage Inducibility

A plate containing the sample for the preparation of comet specimen after completion of the treatment in (4) is centrifuged (1500 rpm, 5 min) and the supernatant is substituted with Hanks' Balanced Salt Solution (HBSS) containing 20 mM EDTA, 10% DMSO (the solution is hereinafter indicated as homogenizing solution). The plate is centrifuged again (1500 rpm, 5 min) and the supernatant is substituted with a homogenizing solution. The cell suspension is mixed with 9-fold volume (v/v) of low melting point agarose solution (PBS containing 0.5% Nu Sieve GTG Agarose), added and spread on the MAS coat slide, and solidified.

The slide is immersed in cell lysis solution (2.5 M sodium chloride, 100 mM EDTA, 10 mM Tris, 10% DMSO, 1% Triton-X100 aqueous solution (pH10)) at 4° C. overnight to lyse cellular membrane and nuclear envelope. The slide after lysis is stood in an electrophoresis solution (0.3 M sodium hydroxide, 1 mM EDTA aqueous solution (pH>13)) for 20 min and electrophoresed at constant voltage of 26 V (0.7 V/cm), 300 mA, 4° C. for 20 min. The slide after electrophoresis is neutralized by immersing in 0.4 M Tris aqueous solution (pH 7.5), sufficiently dehydrated in ethanol, and air dried to give a specimen. Two specimens are prepared for 1 condition.

The prepared specimen is stained for nucleic acid using SYBR (registered trade mark) Gold, and observed under a fluorescence microscope at 200-fold magnification. Not less than 50 cell nuclei per specimen and not less than 100 cell nuclei per condition are imaged, % tail DNA is calculated using an image analyzer Comet Assay IV, and DNA damaging activity is determined. For conditions under which 100 nuclei cannot be observed due to cytotoxicity of the test substance, all nuclei on a slide glass are observed.

The % tail DNA is calculated as a percentage of the fluorescence brightness of the tail to the fluorescence brightness of the whole cell nucleus. Median % tail DNA of total observed nuclei (50 or more (e.g., 75)) is taken per specimen, mean of median of % tail DNA obtained from two pieces of specimen for one condition is taken as the measured value of % tail DNA under this condition, whereby an index of DNA damage by the test substance is obtained.

In addition, a bell-shaped nucleus (hedgehog) recognized as a sign of apoptosis due to cytotoxicity of the test substance is excluded from the calculation targets of % tail DNA.

Dunnett's test is performed between the % tail DNA in a test substance treatment group and the % tail DNA in the corresponding negative control group, and the presence or absence of a significant increase with significance probability (two-sided, 5% or 1%) is tested.

As an index of the cytostasis by a test substance, namely, cytotoxicity, RICC (Relative increase in cell count) is calculated according to the following formula and based on the number of cells at the start of the test substance treatment and at the completion of culture, which are measured in (4):

$$R I C C ( \% ) = \frac{( \begin{matrix}cell number at completion of culture in test substance \text{-} \\ treated group - cell number at start of treatment\end{matrix} )}{( \begin{matrix}cell number at completion of culture in negative \\ control group - cell number at start of treatment\end{matrix} )} \times 100$$

For respective test substances, the case when a 5% significant increase is not found in % tail DNA at all treatment doses of RICC of not less than 40% is judged as negative, and the case when a 5% significant increase is found in % tail DNA at a treatment dose of RICC of not less than 40% is judged as positive. A treatment dose of RICC of less than 40% is excluded from the determination of the DNA damaging activity because an increase in the % tail DNA with low biological significance is likely to be found due to cytotoxicity.

TABLE 6
List of test substance treatment dose used for in vitro comet assay

| test substance | treatment dose [μg/mL] |
|---|---|
| EMS | 62.5, 125, 250, 500 |
| MAN | 50, 100, 200 |

### Example 5 In Vitro Chromosomal Aberration Test Using Human iPS Cell-Derived T Lymphocyte

### (1) Human iPS Cell-Derived T Lymphocyte

As human iPS cell-derived T lymphocyte, redifferentiated T lymphocyte derived from human iPS cell line “2” described in Example 1(1) was used.

### (2) Test Substance

As a test substance, MMC and EMS were used as compounds that induce chromosomal structural aberration and MAN was used as a compound that does not induce chromosomal structural aberration. A test substance was used at the treatment dose (final concentration in medium) described in Table 7. For the treatment, a test substance was dissolved in PBS at a 100-fold concentration of the final concentration in the medium for addition at 1% (v/v). Treatment with a test substance was performed according to the process described in (4).

### (3) Proliferation Stimulation Method

For proliferation stimulation of redifferentiated T lymphocytes, a 24 well plate was used after coating with an anti-CD3 antibody when in use. 0.5 g/mL Anti-Human CD3 Functional Grade Purified (0.5 mL) was added to a 24 well plate, and the plate was stood at room temperature for about 9 hr to coat the plate surface with the antibody. Each well was washed twice with 1 mL of PBS, redifferentiated T lymphocytes derived from human iPS cell line described in (1) were suspended in L-glutamine-containing RPMI-1640 medium (Wako) supplemented with 5% human serum AB (Nova Biologics), 1% Penicillin-Streptomycin (nacalai tesque), 10 ng/mL IL-7 (Wako), and 10 ng/mL IL-15 (R&D SYSTEMS) (hereinafter to be indicated as Rh-5 medium) at a cell density of 2.0×10⁶ cells/mL and added to each well. After about 14 hr of culture, the total amount of the cell suspension was recovered by pipetting. The cell suspension was diluted with Rh-5 medium to a cell density of 2.0×10⁵ cells/mL and reseeded in a 12 well plate not coated with the antibody at 2 mL per well, and culture was continued.

### (4) Test Substance Treatment Method

Treatments with MMC, EMS and MAN were performed according to the method described below. That is, after about 24 hr from the reseeding in (3), a solution (20 μL) obtained by dissolving a test substance in PBS at a 100-fold concentration of the final concentration described in Table 5 was further added and the cells were treated for 27 hr. Different from a test substance treatment group, PBS (20 μL) was added and a similar treatment was performed for 27 hr to prepare a negative control group. About 2 hr before completion of the treatment, KaryoMAX (registered trade mark) Colcemid™ Solution in HBSS (Life technologies) (20 μL) was added to the well (final concentration in medium: 0.1 μg/mL) to discontinue the cell cycle, and cells in mitotic metaphase were enriched.

At the time of start of the test substance treatment, a part of the cell suspension of the negative control group was separated and the number of the cells was measured using a counting chamber.

### (5) Specimen Preparation Method and Evaluation Method of Chromosomal Aberration Inducibility

A part of the cell suspension after the treatment and completion of culture in (4) was separated, and the number of cells was measured using a counting chamber. All remaining cell suspension was collected in a centrifuge tube and PBS was added to a total volume of 5 mL, after which the mixture was centrifuged (1000 rpm, 5 min). The supernatant was removed, 75 mM KCl heated to 37° C. was added and a hypotonic treatment was performed for 5 min. To the cell suspension after the hypotonicity was added 1/5 volume of a fixation solution (methanol:glacial acetic acid=3:1), and the cells were semi-fixed and centrifuged (4° C., 1000 rpm, 3 min). After centrifugation, the supernatant was substituted with a fresh fixation solution, and the cells were fixed and centrifuged (4° C., 1000 rpm, 3 min). The operation was repeated 3 times, and the cells were sufficiently fixed and centrifuged again (4° C., 1000 rpm, 3 min). The cells were suspended in a small amount of a fixation solution. The suspension was added dropwise on a slide glass and air dried to give a specimen.

The prepared specimen was stained with Giemsa and observed under a stereomicroscope at 1000-fold magnification. The chromosomes were observed in 300 cells in mitotic metaphase having well-spread chromosomes per condition to determine the presence or absence of structural aberration of the chromosome.

Chromosomal abnormalities were classified into chromatid break, chromosome break, chromatid exchange, chromosome exchange, and other structural abnormalities and counted. Of the chromatid or chromosome break, when the unstained site was smaller than the width of the chromatid and did not deviate from the axis of the chromatid, the unstained site was a chromatid or chromosome gap and was not included in the chromosomal aberration.

As an index of chromosomal aberration inducibility by a test substance, frequency of chromosomal aberration was obtained by dividing the number of cells in mitotic metaphase with any chromosomal aberration by the total number of observed cells in mitotic metaphase. In addition, the Fisher's exact test was performed between the frequency of chromosomal aberration in a test substance treatment group and the frequency of chromosomal aberration in the corresponding negative control group, and the presence or absence of a significant increase with significance probability (two-sided, 5% or 1%) was tested.

As an index of the cytostasis by a test substance, namely, cytotoxicity, RICC (Relative increase in cell count) was calculated according to the following formula and based on the number of cells at the start of the test substance treatment and at the completion of culture, which were measured in (4):

$$R I C C ( \% ) = \frac{( \begin{matrix}cell number at completion of culture in test substance \text{-} \\ treated group - cell number at start of treatment\end{matrix} )}{( \begin{matrix}cell number at completion of culture in negative \\ control group - cell number at start of treatment\end{matrix} )} \times 100$$

For respective test substances, the case when a 5% significant increase was not found in frequency of chromosomal aberration at all treatment doses of RICC of not less than 30% was judged as negative, and the case when a 5% significant increase was found in frequency of chromosomal aberration at a treatment dose of RICC of not less than 30% was judged as positive.

A treatment dose of RICC of less than 30% was excluded from the determination of the chromosomal aberration inducibility because an increase in the frequency of chromosomal aberration with low biological significance was likely to be found due to cytotoxicity.

### (6) Results

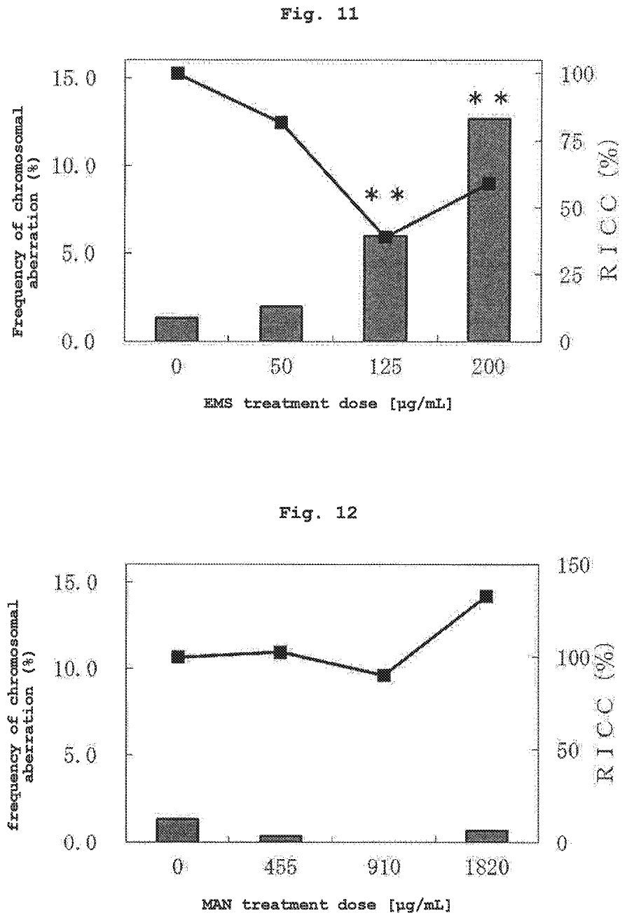

The judgment results obtained in this Example for the three compounds as test compounds and the judgment results expected from known information (Kirkland, D. et al. Mutation Research 2016, 795, 7-30; Lasne, C. et al. Mutation Research 1984, 130(4), 273-282) are shown in Table 8. In addition, the dose response relationships of respective test substances are shown in FIG. 10-FIG. 12.

As shown in Table 8 and FIG. 10-FIG. 12, the responsiveness expected from known information could be correctly detected for all of the three compounds as test compounds including a chromosomal aberration inducible compound and a chromosomal aberration non-inducible compound. That is, it was demonstrated that the in vitro chromosomal aberration test using human iPS cell-derived T lymphocyte can be practically used for the evaluation of chromosomal aberration inducibility of a test substance.

TABLE 7
List of test substance treatment dose used
for in vitro chromosomal aberration test

| test substance | treatment dose | |
|---|---|---|
| MMC | 0.0125, 0.025, 0.05 | μg/mL |
| EMS | 50, 125, 200 | μg/mL |
| MAN | 455, 910, 1820 | μg/mL |

TABLE 8
Outline of results of in vitro chromosomal aberration
test using human iPS cell-derived T lymphocyte

| test | judgment result obtained | judgment result expected |
|---|---|---|
| substance | in this Example | from known information |
| MMC | positive | positive |
| EMS | positive | positive |
| MAN | negative | negative |

### Example 6 In Vitro Comet Assay Using Human iPS Cell-Derived T Lymphocyte

### (1) Human iPS Cell-Derived T Lymphocyte

As human iPS cell-derived T lymphocyte, redifferentiated T lymphocyte derived from human iPS cell line “2” described in Example 1(1) was used.

### (2) Test Substance

As a test substance, EMS was used as a compound that induces DNA damage and MAN was used as a compound that does not induce DNA damage. A test substance was used at the treatment dose (final concentration in medium) described in Table 9. For the treatment, a test substance was dissolved in PBS at a 100-fold concentration of the final concentration in the medium for addition at 1% (v/v). A test substance was treated according to the process described in (4).

### (3) Proliferation Stimulation Method

For proliferation stimulation of redifferentiated T lymphocytes, a 24 well plate was used after coating with an anti-CD3 antibody when in use. 0.5 μg/mL Anti-Human CD3 Functional Grade Purified (0.5 mL) was added to a 24 well plate, and the plate was stood at room temperature for about 10 hr to coat the plate surface with the antibody. Each well was washed twice with 1 mL of PBS, redifferentiated T lymphocytes derived from human iPS cell line described in (1) were suspended in Rh-medium at a cell density of 2.0×10⁶ cells/mL and added to each well. After about 13 hr of culture, the total amount of the cell suspension was recovered by pipetting. The cell suspension was diluted with Rh-5 medium to a cell density of 2.0×10⁵ cells/mL and reseeded in a 24 well plate not coated with the antibody at 1 mL per well, and culture was continued.

### (4) Test Substance Treatment Method

Treatments with EMS and MAN were performed according to the method described below. That is, after about 24 hr from the reseeding in (3), a solution (10 μL) obtained by dissolving a test substance in PBS at a 100-fold concentration of the final concentration described in Table 9 was further added and the cells were treated for 4 hr. Different from a test substance treatment group, PBS (10 μL) was added and a similar treatment was performed for 4 hr to prepare a negative control group. The above samples for the preparation of comet specimens were processed in triplicate per condition.

Different from the samples for the preparation of comet specimens, as a sample for cytotoxicity evaluation, treatments with EMS, MAN and PBS were similarly performed for 4 hr. As for the sample for cytotoxicity evaluation, after the treatment for 4 hr, the cells were washed with PBS and reseeded, and additionally cultured for about 28 hr. On completion of the additional culture, a part of the cell suspension was separated and the number of the cells was measured using a counting chamber.

### (5) Specimen Preparation Method and Evaluation Method of DNA Damage Inducibility

The sample for the preparation of comet specimen after completion of the treatment in (4) was recovered in a centrifuge tube and centrifuged (1000 rpm, 5 min) and the supernatant was substituted with Hanks' Balanced Salt Solution (HBSS) containing 20 mM EDTA, 10% DMSO (the solution is hereinafter indicated as homogenizing solution). The sample was centrifuged again (1000 rpm, 5 min) and the supernatant was substituted with a homogenizing solution. The cell suspension was mixed with 9-fold volume (v/v) of low melting point agarose solution (PBS containing 0.5% Nu Sieve GTG Agarose), added and spread on the MAS coat slide, and solidified.

The slide was immersed in cell lysis solution (2.5 M sodium chloride, 100 mM EDTA, 10 mM Tris, 10% DMSO, 1% Triton-X100 aqueous solution (pH10)) at 4° C. overnight to lyse cellular membrane and nuclear envelope. The slide after lysis was stood in an electrophoresis solution (0.3 M sodium hydroxide, 1 mM EDTA aqueous solution (pH>13)) for 20 min and electrophoresed at constant voltage of 26 V (0.7 V/cm), 300 mA, 4° C. for 20 min. The slide after electrophoresis was neutralized by immersing in 0.4 M Tris aqueous solution (pH 7.5), sufficiently dehydrated in ethanol, and air dried to give a specimen. Three specimens were prepared for 1 condition per 1 series.

The prepared specimen was stained for nucleic acid using SYBR (registered trade mark) Gold, and observed under a fluorescence microscope at 200-fold magnification. 75 nuclei per specimen and 150 nuclei in two specimens per condition were imaged, % tail DNA was calculated using an image analyzer Comet Assay IV, and DNA damaging activity was determined.

The % tail DNA was calculated as a percentage of the fluorescence brightness of the tail to the fluorescence brightness of the whole cell nucleus. Median % tail DNA of total observed nuclei (e.g., 75) was taken per specimen, mean of median of % tail DNA obtained from two pieces of specimen for one condition per series was taken as the measured value of % tail DNA under this condition, whereby an index of DNA damaging activity by the test substance was obtained. In addition, a bell-shaped nucleus (hedgehog) recognized as a sign of apoptosis due to cytotoxicity of the test substance was excluded from the calculation targets of % tail DNA.

Dunnett's test was performed between the % tail DNA in a test substance treatment group and the % tail DNA in the corresponding negative control group, and the presence or absence of a significant increase with significance probability (two-sided, 5% or 1%) was tested.

As an index of the cytostasis by a test substance, namely, cytotoxicity, RCC (Relative cell count) was calculated according to the following formula and based on the number of cells at the completion of culture, which was measured in (4):

$$R C C ( \% ) = \frac{\begin{matrix}cell num ber at completion of culture in \\ test substance \text{-} treated group\end{matrix}}{\begin{matrix}cell number at completion of culture in \\ negative control group\end{matrix}} \times 100$$

For respective test substances, the case when a 5% significant increase was not found in % tail DNA at all treatment doses of RCC of not less than 30% was judged as negative, and the case when a 5% significant increase was found in % tail DNA at a treatment dose of RCC of not less than 30% was judged as positive. A treatment dose of RCC of less than 30% was excluded from the determination of the DNA damaging activity because an increase in the % tail DNA with low biological significance was likely to be found due to cytotoxicity.

### (6) Results

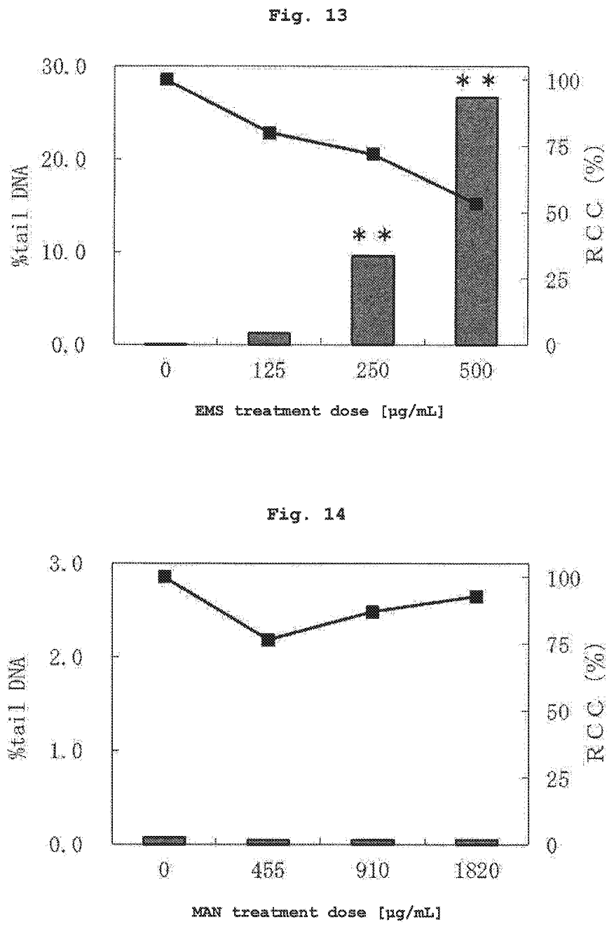

The judgment results obtained in this Example for EMS and MAN and the judgment results expected from known information (Kirkland, D. et al. Mutation Research 2016, 795, 7-30; Lasne, C. et al. Mutation Research 1984, 130(4), 273-282) are shown in Table 10. In addition, the dose response relationships of respective test substances are shown in FIG. 13 and FIG. 14.

As shown in Table 10, FIG. 13 and FIG. 14, for both DNA damage inducible compound EMS and DNA damage non-inducible compound MAN, responsiveness expected from known information could be correctly detected. That is, it was demonstrated that the in vitro comet assay using human iPS cell-derived T lymphocyte can be practically used for the evaluation of DNA damage inducibility of a test substance.

TABLE 9
List of test substance treatment dose used for in vitro comet assay

| test substance | treatment dose | |
|---|---|---|
| EMS | 125, 250, 500 | μg/mL |
| MAN | 455, 910, 1820 | μg/mL |

TABLE 10
Outline of results of in vitro comet assay
using human IPS cell-derived T lymphocyte

| test | judgment result obtained | judgment result expected |
|---|---|---|
| substance | in this Example | from known information |
| EMS | positive | positive |
| MAN | negative | negative |

### Comparative Example 1 Proliferation Ability of Human iPS Cell-Derived T Lymphocyte by Various Proliferation Stimulation Factors

### (1) Human iPS Cell-Derived T Lymphocyte

As human iPS cell-derived T lymphocyte, redifferentiated T lymphocyte derived from human iPS cell line “2” described in Example 1(1) was used. Proliferation stimulation by various proliferation stimulation factors was applied to redifferentiated T lymphocytes and the proliferation ability thereof was evaluated by measuring the ATP amount.

### (2) Evaluation Method of Proliferation Ability

Redifferentiated T lymphocytes were seeded at a cell density of 1.0×10⁵ cells/mL on a 96 well plate (nunc) at 100 μL per well and cultured in the following 7 kinds of media. That is, redifferentiated T lymphocytes were cultured in the following 7 kinds of media of (a) Rh medium without addition of proliferation stimulation factor (no stimulation), (b) Rh medium supplemented with 5 μg/mL PHA (Sigma-Aldrich), (c) Rh medium supplemented with 1 μg/mL PHA, (d) Rh medium supplemented with 100 U/mL IL-2, (e) Rh medium supplemented with 25 ng/mL phorbol 12-myristate 13-acetate (PMA) (Wako) and 1 μg/mL Ionomycin (Wako), (f) Rh medium supplemented with 50 ng/mL PMA, (g) Rh medium supplemented with 5 μg/mL Con A (Sigma-Aldrich), and proliferation stimulation was applied for 66 hr. After completion of the stimulation period, each well was sufficiently pipetted and the cell suspension was separated in a white 96 well plate (nunc) at 50 μL per well. The ATP amount was measured using CellTiter-Glo™ Luminescent Cell Viability Assay Kit (Promega) and used as an index of the number of viable cells. The ATP amount was measured according to the instruction manual of the Kit. That is, CellTiter-Glo (registered trade mark) Substrate dissolved in CellTiter-Glo (registered trade mark) Buffer was added at 50 μL per well and the mixture was stirred using a shaker under shading at room temperature for 15 min. After stirring, the luminescence signal of each well was measured using a plate reader EnVision (PerkinElmer). When seeding the redifferentiated T lymphocytes, 50 μL of the cell suspension was separately taken in a white 96 well plate and the ATP amount was similarly measured using the CellTiter-Glo™ Luminescent Cell Viability Assay.

### (3) Results

The results are shown in Table 11. Table 11 shows, as the luminescence signal of each well, a value obtained by subtracting the count value of the background luminescence signal simultaneously measured for Rh medium from the count value of the luminescence signal measured for each well. Also, the percentage of each luminescence signal to the luminescence signal at the time of seeding is shown as a proliferation rate.

From Table 11, it was shown that, under any proliferation stimulation conditions used in this Comparative Example, redifferentiated T lymphocyte does not proliferate but rather, the cell number thereof decreases from that at the time of seeding.

TABLE 11
Proliferation ability of human iPS cell-derived T lymphocyte
by various proliferation stimulation factors

| proliferation stimulation | luminescent | proliferation |
|---|---|---|
| condition | signal (count) | rate |
| on seeding | 128480 | 100.0% |
| no stimulation | 8704 | 6.8% |
| 5 μq/mL PHA | 15024 | 11.7% |
| 1 μg/mL PHA | 10984 | 8.5% |
| 100 U/mL IL-2 | 11784 | 9.2% |
| 25 ng/mL PMA + 1 μg/mL | 26304 | 20.5% |
| Ionomycin | | |
| 50 ng/mL PMA | 5784 | 4.5% |
| 5 μg/mL Con A | 10064 | 7.8% |

### Comparative Example 2 Division Kinetics of Human iPS Cell-Derived T Lymphocyte Stimulated with PHA

### (1) Human iPS Cell-Derived T Lymphocyte

As human iPS cell-derived T lymphocyte, redifferentiated T lymphocyte derived from human iPS cell line “2” described in Example 1(1) was used. Redifferentiated T lymphocytes were subjected to proliferation stimulation with PHA under coculture with feeder cells or in the absence of feeder cells. The cytokinesis inhibitor CytoB was added, specimens were prepared and the distribution of the number of nuclei per cell was measured, based on which the division kinetics thereof was evaluated.

### (2) Proliferation Stimulation Conditions

<PHA Stimulation Conditions Under Coculture with Feeder Cells>

As the feeder cell, human peripheral blood-derived mononuclear cells PBMC (peripheral blood mononuclear cells) with cell division discontinued by 35Gy X-ray radiation were used. The feeder cells (4.0×10⁵ cells) and redifferentiated T lymphocytes (5.0×10⁴ cells) were mixed, suspended in Rh medium (100 μL) supplemented with 5 μg/mL PHA, and the total amount was seeded on a 96 well plate (Techno Plastic Products AG) (cell density: feeder cell 4.0×10⁶ cells/mL, redifferentiated T lymphocyte 5.0×10⁵ cells/mL). After culture for about 44 hr, Rh medium (11.1 μL) supplemented with 60 μg/mL CytoB and 5 μg/mL PHA was added to the well (final concentration of CytoB: 6 μg/mL). After further culture for about 24 hr, specimens were prepared according to the process described in (3). As a comparison control, a specimen was prepared by similarly applying PHA stimulation to feeder cells (4.0×10⁵ cells, cell density: 4.0×10⁶ cells/mL) without mixing redifferentiated T lymphocytes and adding CytoB.

### <PHA Stimulation Conditions in the Absence of Feeder Cells>

Redifferentiated T lymphocytes (7.9×10⁴ cells) were suspended in Rh medium (100 μL) supplemented with 3 μg/mL PHA, and the total amount was seeded on a 96 well plate (Techno Plastic Products AG) (cell density: 7.9×10⁵ cells/mL). After culture for about 47 hr, Rh medium (11.1 μL) supplemented with 60 μg/mL CytoB was added to the well (final concentration of CytoB: 6 μg/mL). After further culture for about 24 hr, specimens were prepared according to the process described in (3).

### (3) Specimen Preparation Method and Division Kinetics Evaluation Method

The plate after completion of culture in (2) was fixed according to the method described in Example 2(5), and a specimen was prepared on a slide glass. The prepared specimen was stained with 40 μg/mL acridine orange solution and observed under a fluorescence microscope using a wide-band Blue excitation filter. 500 cells were observed per specimen and the numbers of mononucleated cells, binucleated cells and multinucleated cells were measured. However, the specimen subjected to PHA stimulation in the absence of feeder cells showed an extremely small cell number and failed to secure 500 cells for observation. Therefore, the entire 426 cells on the slide glass were observed.

As an index of division kinetics, CBPI was calculated in the same manner as in Example 1(3).

### (4) Results

The results are shown in Table 12.

Since CBPI with the feeder cells was 1.03, discontinuation of cell division of the feeder cell by X-ray irradiation could be confirmed. On the other hand, since CBPI of the redifferentiated T lymphocytes subjected to PHA stimulation under coculture with the feeder cells was 1.15, induction of cell division of the redifferentiated T lymphocytes was suggested. However, it was shown that human iPS cell-derived T lymphocytes divided at sufficient frequency cannot be observed due to the presence of a large amount of the mixed feeder cells. In addition, it was shown that the redifferentiated T lymphocyte subjected to PHA stimulation in the absence of feeder cells showed CBPI of 1.39, and thus the PHA stimulation fails to sufficiently induce cell division.

That is, it was shown that, with the proliferation stimulation by PHA, human iPS cell-derived T lymphocytes divided at sufficient frequency cannot be observed irrespective of the presence or absence of coculture with feeder cells, and the cells are not practical for detection or test of chromosomal aberration.

TABLE 12
Division kinetics of human iPS cell-derived
T lymphocyte by stimulation with PHA

| | | mono- | bi- | multi- | |
|---|---|---|---|---|---|
| | observed | nucleated | nucleated | nucleated | |
| | cell | cell | cell | cell | |
| specimen | number | number | number | number | CBPI |
| feeder cell | 500 | 488 | 11 | 1 | 1.03 |
| feeder cell + re- | 500 | 426 | 71 | 3 | 1.15 |
| differentiated | | | | | |
| T lymphocyte | | | | | |
| redifferentiated | 426 | 262 | 160 | 4 | 1.39 |
| T lymphocyte | | | | | |

From the foregoing Examples, it was shown from Comparative Example 1 that human iPS cell-derived T lymphocyte does not proliferate by proliferation stimulation for 66 hr with any of the proliferation stimulation factors of PHA, IL-2, phorbol 12-myristate 13-acetate (PMA), Ionomycin and Concanavalin A (Con A) but rather, the viable cell number decreases markedly from that after the stimulation. In addition, from the results of Comparative Example 2 in which short-term cell division kinetics were confirmed using cytokinesis inhibitor CytoB, it was shown that, with the proliferation stimulation by PHA, human iPS cell-derived T lymphocytes divided at sufficient frequency cannot be observed irrespective of the presence or absence of coculture with feeder cells, and the cells are not practical for detection or test of chromosomal aberration.

On the other hand, from Example 1, it was shown that human iPS cell-derived T lymphocyte cell shows good division kinetics by proliferation stimulation with an anti-CD3 antibody, and human iPS cell-derived T lymphocytes divided at sufficient frequency can be observed. Furthermore, from Example 2, it was shown by the in vitro micronucleus test using human iPS cell-derived T lymphocytes that the reactivity of known micronucleus inducible compounds and micronucleus non-inducible compounds can be appropriately evaluated. In addition, from Example 5, it was shown by the in vitro chromosomal aberration test using human iPS cell-derived T lymphocytes that the reactivity of known chromosomal aberration inducible compounds and chromosomal aberration non-inducible compounds can be appropriately evaluated. Furthermore, from Example 6, it was shown by the in vitro comet assay using human iPS cell-derived T lymphocytes that the responsiveness of known DNA damage inducible compounds and DNA damage non-inducible compounds can be appropriately evaluated.

That is, detection or testing cannot be performed by proliferation stimulation using PHA, which is adopted in the conventional methods by HuLy, since human iPS cell-derived T lymphocyte does not divide sufficiently. However, when an anti-CD3 antibody is used as a proliferation stimulation factor, T lymphocyte is found to divide well and detection and testing can be performed. Also, detection and testing of chromosomal aberration using human pluripotent stem cell-derived T lymphocyte has been established.

This application is based on a patent application No. 2016-252673 filed in Japan (filing date: Dec. 27, 2016), the contents of which are incorporated in full herein.

### INDUSTRIAL APPLICABILITY

As described above, by using human pluripotent stem cell-derived T lymphocytes, chromosomal abnormalities can be detected using human normal cell type that can be stably and homogeneously supplied in large amounts. As a result, refinement and efficiency of the evaluation of chromosomal aberration inducibility of chemical substance and so on can be realized as a next-generation standard test system that solves the problems of conventional cell types. Furthermore, high quality of human safety evaluation can be realized in high precision analysis of human individual differences in cell response and so on, which was difficult with conventional techniques.

1. A method for detecting chromosomal aberration comprising the following steps (1) and (2):
(1) a step of adding an anti-CD3 antibody to a human pluripotent stem cell-derived T lymphocyte and culturing the T lymphocyte,
(2) a step of detecting chromosomal aberration in the T lymphocyte cultured in step (1).

2. The detection method according to claim 1 wherein the human pluripotent stem cell is a human ES cell.

3. The detection method according to claim 1 wherein the human pluripotent stem cell is a human iPS cell.

4. The detection method according to claim 1 wherein step for detecting chromosomal aberration is a step for detecting chromosomal aberration by measuring frequency of micronucleus.

5. The detection method according to claim 1 wherein the step for detecting chromosomal aberration is a step for detecting chromosomal aberration by observation of chromosomal morphology.

6. The detection method according to claim 1 wherein the step for detecting chromosomal aberration is a step for detecting chromosomal aberration by evaluation of DNA damage.

7. A detection kit for use in the detection method according to claim 1 comprising an anti-CD3 antibody and a human pluripotent stem cell-derived T lymphocyte.

8. A method for evaluating inducibility by a test substance of chromosomal aberration comprising the following steps (1)-(4):
(1) a step of adding an anti-CD3 antibody to a human pluripotent stem cell-derived T lymphocyte and culturing the T lymphocyte,
(2) a step of adding a test substance to the T lymphocyte cultured in step (1),
(3) a step of measuring frequency of chromosomal aberration in the T lymphocyte after addition of the test substance,
(4) a step of evaluating inducibility of chromosomal aberration by comparing the frequency of chromosomal aberration with the standard value.

9. The method according to claim 8 wherein the human pluripotent stem cell is a human ES cell.

10. The method according to claim 8 wherein the human pluripotent stem cell is a human iPS cell.

11. The method according to claim 8 wherein step (3) is a step of measuring frequency of chromosomal aberration in T lymphocyte after addition of a test substance by measuring frequency of micronucleus.

12. The method according to claim 8 wherein step (3) is a step of measuring frequency of chromosomal aberration in T lymphocyte after addition of a test substance by observation of chromosomal morphology.

13. The method according to claim 8 wherein step (3) is a step of measuring frequency of chromosomal aberration in T lymphocyte after addition of a test substance by evaluation of DNA damage.

14. A test kit for use in the evaluation method according to claim 8, comprising an anti-CD3 antibody and a human pluripotent stem cell-derived T lymphocyte.
